(19) United States
(12) Patent Application Publication
(10) Pub. No.: US 2025/0387629 A1
(43) Pub. Date: Dec. 25, 2025

(54) IMPLANTABLE MEDICAL DEVICE

(71) Applicant: Medtronic, Inc., Minneapolis, MN (US)

(72) Inventors: Walton W. Baxter, III, San Clemente, CA (US); Kevin T. O'Brien, Arden Hills, MN (US); Kevin L. Sack, New Brighton, MN (US); William J. Clemens, Fridley, MN (US); Jonathan E. Baxter, Fridley, MN (US)

(21) Appl. No.: 19/240,063

(22) Filed: Jun. 17, 2025

Related U.S. Application Data
(60) Provisional application No. 63/661,791, filed on Jun. 19, 2024.

(51) Int. Cl. A61N 1/375; A61N 1/05; A61N 1/362

(52) U.S. Cl. CPC A61N 1/3756; A61N 1/0573; A61N 1/362; A61N 1/37512

(57) ABSTRACT

An implantable medical device (IMD) configured to position within a heart of a patient. The IMD may include a primary helix supporting a first electrode and a secondary helix supporting a second electrode. The primary helix is configured to travel into a tissue wall when the IMD is rotated in a first rotational direction and withdraw from the tissue wall when the IMD is rotated in a second rotational direction opposite the first rotational direction. The secondary helix is configured to travel into the tissue wall when the IMD is rotated in the second rotational direction and withdraw from the tissue wall when the IMD is rotated in the first rotational direction. In examples, the IMD includes a support member configured to assist the secondary helix in penetrating the tissue wall when the IMD rotates in the second rotational direction.

### RELATED APPLICATIONS

This application claims the benefit of U.S. Provisional Application Ser. No. 63/661,791, filed Jun. 19, 2024, the entire contents of each of which are incorporated herein by reference.

### TECHNICAL FIELD

This disclosure is related to an implantable medical device.

### BACKGROUND

Implantable medical devices are often placed in a subcutaneous pocket and coupled to one or more transvenous medical electrical leads carrying pacing and sensing electrodes positioned in the heart. Intracardiac pacemakers have recently been introduced that are implantable within a ventricular chamber of a patient's heart for delivering ventricular pacing pulses without the use of electrical leads. Such pacemakers or other implantable medical devices may also be able to detect the occurrence of arrhythmias, such as fibrillation, tachycardia and bradycardia, in the patient's heart. An implantable cardiac defibrillator may deliver electrical shocks to the patient's heart in response to detection of a tachycardia or fibrillation to restore a normal heartbeat in the patient. In some cases, a single implantable medical device functions as both an implantable pacemaker and implantable cardiac defibrillator.

Implantable medical devices may include electrodes and/or other elements for physiological sensing and/or therapy delivery. The electrodes and/or other elements may be implanted at target locations selected to detect a physiological condition of the patient and/or deliver one or more therapies. For example, the electrodes and/or other elements may be delivered to a target location within an atrium or ventricle to sense intrinsic cardiac signals and deliver pacing or antitachyarrhythmia shock therapy from a medical device coupled to a lead.

### SUMMARY

This disclosure describes an implantable medical device (IMD) configured to position within a heart of a patient. The IMD may be configured to implant at least one of a first electrode or a second electrode within tissue of a patient, such as a septal wall of the heart. The IMD is configured to position within a heart of a patient, such as within an atrium, ventricle, coronary sinus, or other portions of the heart. The IMD may include a primary helix supporting the first electrode and a secondary helix supporting the second electrode. The primary helix is configured to travel into the tissue wall when the IMD is rotated in a first rotational direction and withdraw from the tissue wall when the IMD is rotated in a second rotational direction opposite the first rotational direction. The secondary helix is configured to travel into the tissue wall when the IMD is rotated in the second rotational direction and withdraw from the tissue wall when the IMD is rotated in the first rotational direction. Hence, the IMD is configured such that an insertion depth of the first electrode and an insertion depth of the second electrode within the tissue wall may be controlled (e.g., by a clinician) using a direction of rotation of the IMD.

In an example, a medical device configured to be positioned within an anatomical volume defined by a body of a patient comprises: a primary helix surrounding a longitudinal axis defined by a housing of the medical device, wherein the primary helix extends in a distal direction beyond a distal portion of the housing, wherein the primary helix supports a first electrode, and wherein the primary helix is configured to displace the first electrode within a tissue wall of a patient when a torque in a first rotational direction about the longitudinal axis is imparted to a housing; a secondary helix surrounding the longitudinal axis, wherein the secondary helix extends in the distal direction beyond the distal portion of the housing, wherein the secondary helix supports a second electrode, and wherein the secondary helix is configured to displace the second electrode within the tissue wall when a torque in a second rotational direction about the longitudinal axis is imparted to the housing; and processing circuitry electrically connected to the first electrode and the second electrode, wherein the processing circuitry is configured to at least one of provide a signal or sense a signal using at least one of the first electrode or the second electrode.

In an examples, a medical device configured to be positioned within an anatomical volume defined by a body of a patient comprises: a primary helix surrounding a longitudinal axis defined by a housing of the medical device, wherein the primary helix extends in a distal direction beyond a distal portion of the housing, and wherein the primary helix is configured to displace a distal end of primary helix within a tissue wall of a patient when a torque in a first rotational direction about the longitudinal axis is imparted to a housing; a secondary helix surrounding the longitudinal axis, wherein the secondary helix extends in the distal direction beyond the distal portion of the housing, and wherein the secondary helix is configured to displace a distal end of the secondary helix within the tissue wall when a torque in a second rotational direction about the longitudinal axis and opposite the first rotational direction is imparted to the housing, wherein at least one of the primary helix or the secondary helix supports an electrode; a support member supported by the housing, wherein the support member is configured to establish at least the distal end of the secondary helix distal to a distal end of the housing when a force in the proximal direction is imparted on the secondary helix; and processing circuitry electrically connected to the electrode, wherein the processing circuitry is configured to at least one of provide a signal or sense a signal using the electrode.

In an example, a method comprises: displacing, using a housing of a medical device, a first electrode within a tissue wall of a patient by imparting, to a primary helix supported by the housing, a torque in a first rotational direction about a longitudinal axis defined by the housing, wherein the primary helix extends in a distal direction beyond a distal portion of the housing, and wherein the primary helix supports the first electrode; displacing, using the housing, a second electrode within the tissue wall by imparting, to a secondary helix supported by the housing, a torque in a second rotational direction about the longitudinal axis, wherein the secondary helix extends in the distal direction beyond the distal portion of the housing, and wherein the secondary helix supports the second electrode; and at least one of providing or sensing, using processing circuitry, a signal using at least one of the first electrode or the second electrode.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system including an implantable medical device.

FIG. 2 is a schematic plan view of the implantable medical device.

FIG. 3 is a schematic end view of the implantable medical device of FIG. 2.

FIG. 4 is a schematic illustration of a primary helix and a secondary helix of the implantable medical device in a first configuration relative to a tissue wall.

FIG. 5 is a schematic illustration of the primary helix and the secondary helix of FIG. 4 in a second configuration relative to the tissue wall.

FIG. 6 is a schematic illustration of the primary helix and the secondary helix of FIG. 4 and FIG. 5 in a third configuration relative to the tissue wall.

FIG. 7 is a schematic illustration of the primary helix and the secondary helix of FIG. 4, FIG. 5, and FIG. 6 in a fourth configuration relative to the tissue wall.

FIG. 8 is a schematic illustration of an example support member of the implantable medical device.

FIG. 9 is a schematic illustration of another example of a support member of the implantable medical device.

FIG. 10A is an example of a secondary helix in a first orientation relative to a housing of the implantable medical device.

FIG. 10B is an example of the secondary helix of FIG. 10A in a second orientation relative to the housing of the implantable medical device.

FIG. 11A is a schematic plan view of a secondary helix of the implantable medical device embedded within a tissue wall.

FIG. 11B is a schematic end view of the secondary helix of FIG. 11A.

FIG. 11C is a schematic plan view of the secondary helix of FIG. 11A and FIG. 11B rotated a first amount in a second rotational direction.

FIG. 11D is a schematic end view of the secondary helix of FIG. 11C.

FIG. 11E is a schematic plan view of the secondary helix of FIG. 11A, FIG. 11B, FIG. 11C, and FIG. 11D rotated a second amount in the second rotational direction.

FIG. 11F is a schematic end view of the secondary helix of FIG. 11E.

FIG. 12 illustrates an example technique for using the example implantable medical device.

### DETAILED DESCRIPTION

This disclosure describes an implantable medical device (IMD) configured to implant at least one of a first electrode or a second electrode within tissue of a patient, such as a septal wall of the heart. The IMD is configured to position within a heart of a patient, such as within an atrium, ventricle, coronary sinus, or other portions of the heart. The IMD further includes a primary helix extending from a distal portion of a housing of the IMD and supporting the first electrode. The IMD includes a secondary helix extending from the distal portion of the IMD. The secondary helix may support the second electrode.

The primary helix is configured to displace a distal end of the primary helix (“primary helix distal end”) within the tissue wall when a housing of the IMD (“IMD housing”) is rotated in a first rotational direction. The first rotational direction may be a rotation about a longitudinal axis LD defined by the IMD housing. The secondary helix is configured to displace a distal end of the secondary helix (“secondary helix distal end”) within the tissue wall when the IMD housing is rotated in a second rotational direction opposite the first rotational direction. For example, the primary helix may define one of a right-handed helix or a left-handed helix and the secondary helix may define the other of the right-handed helix or the left-handed helix. In some examples, the tissue wall may be any inner surface of any heart chamber of a patient, including the septum, and/or may be an outer surface of the heart of the patient, and/or may be another tissue wall defined by tissue of the patient.

Hence, the IMD is configured such that a rotation of IMD housing in the first rotational direction causes the primary helix to embed the primary helix distal end within the tissue wall, and a rotation of the IMD housing in the second rotational direction causes the secondary helix to embed the secondary helix distal end within the tissue wall. In examples, the primary helix extends distally beyond the secondary helix, such that the primary helix remains at least partially embedded within the tissue wall when the IMD housing is subsequently rotated in the second rotational direction to embed the secondary helix within the tissue wall.

In examples, the primary helix supports the first electrode and the secondary helix supports the second electrode. The IMD may include processing circuitry configured to deliver therapy to and/or sense signals from a patient using the first electrode and/or the second electrode. The primary helix may be configured to displace the first electrode within the tissue wall when the IMD housing rotated in the first rotational direction. The secondary helix may be configured to displace the second electrode within the tissue wall when the IMD housing is rotated in the second rotational direction. The primary helix and secondary helix may be configured such that a distance in which the first electrode and/or second electrode is embedded within the tissue wall is substantially proportional to an amount and direction of rotation of the IMD housing. Thus, the IMD is configured such that a clinician may control a depth a insertion of the first electrode and/or the second electrode based on a direction of rotation of the IMD housing.

For example, the clinician may impart a torque to the IMD housing in the first rotational direction to cause the primary helix to position the first electrode substantially at a location within the tissue wall sufficient to deliver a first pacing signal to, for example, a conduction system or another portion of the heart 101. The secondary helix (e.g., including the secondary helix distal end) may be configured to slidably translate (e.g., to substantially drag) over a surface of the tissue wall as the clinician imparts torque to the IMD housing in the first rotational direction. The clinician may (e.g., subsequently) impart a torque to the IMD housing in the second rotational direction to cause the secondary helix to position the second electrode substantially at a location within the tissue wall sufficient to deliver a second pacing signal to, for example, atrial tissue or another portion of the heart. The clinician may thus control of a depth of insertion of both the first electrode and the second electrode within the tissue wall using the rotation of the IMD housing in the first rotational direction or the rotation in the second rotational direction.

In examples, the IMD includes a support member configured to assist in causing the secondary helix to penetrate a tissue wall surface of the tissue wall. The support member may be configured to cause at least some portion of the secondary helix (e.g., the secondary helix distal end) to remain distally displaced from the IMD housing. In examples, the support member is configured to cause the secondary helix to remain distally displaced from a distal surface defined by the IMD housing (“IMD distal surface”). The distal displacement between the secondary helix and the IMD housing and/o IMD distal surface may assist in causing the secondary helix distal end to penetrate the tissue wall surface when the IMD housing is rotated in the second rotational direction. The support member may be configured to limit and/or substantially minimize a tendency of the secondary helix distal end to establish a position substantially flush against the distal surface, which might otherwise introduce difficulty in causing the secondary helix distal end to penetrate the tissue wall surface using rotation of the IMD housing in the second rotational direction.

In some examples, the secondary helix is configured to bend and/or alter its orientation with respect to the IMD housing 141 when the torque in the second rotational direction is imparted to the IMD housing. For example, the second may be configured to bend and/or alter its orientation when the secondary helix reaches a threshold travel within the tissue wall (e.g., substantially reaches a limit of its travel into the tissue wall) and the torque continues to be imparted to the IMD housing in the second rotational direction. For example, when both the primary helix and the secondary helix are substantially embedded within the tissue wall 164, the IMD housing might be rotated in the second rotational direction to cause the primary helix to withdraw from the tissue wall. The secondary helix may be configured to bend and/or alter its orientation with respect to the IMD housing and/or the IMD distal surface as the IMD housing rotates in the second rotary direction. The bending and/or alteration of the orientation may result in secondary helix withdrawing from the tissue wall as the primary helix withdraws from the tissue wall (e.g., as the IMD housing is rotated in the second rotational direction).

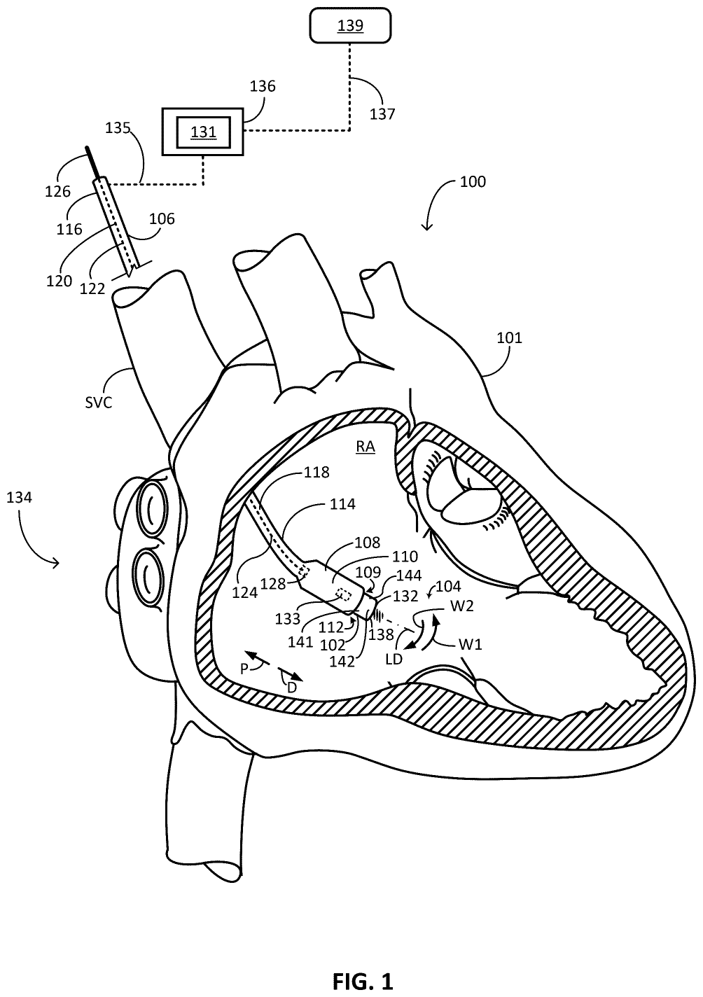

FIG. 1 is a conceptual diagram illustrating an example medical system 100 within a right atrium (“RA”) of a heart 101. Medical system 100 is configured to deliver and/or retrieve an implantable medical device 102 (“IMD 102”) to and/or from the vicinity of a target site 104 of heart 101. Medical system 100 may include a delivery catheter 106 supporting a receptacle device 108. Receptacle device 108 is configured to hold IMD 102 during delivery, deployment, and/or retrieval of IMD 102. Receptacle device 108 includes a receptacle wall 110 defining a receptacle volume 112 configured to hold and/or support IMD 102 during the delivery, deployment, and/or retrieval of IMD 102. In examples, receptacle device 108 defines an opening 109 (“receptacle opening 109”) which opens into receptacle volume 112. Receptacle opening 109 may be configured to allow at least IMD 102 to pass therethrough. In examples, medical system 100 is configured to deploy IMD 102 from receptacle device 108 (e.g., from a position within receptacle volume 112) and through receptacle opening 109 to cause IMD 102 to engage tissues within target site 104. In examples, medical system 100 is configured to cause IMD 102 to disengage from tissues within target site 104 to, for example, retrieve IMD 102 from and/or reposition IMD 102 within heart 101.

Delivery catheter 106 is configured to deliver receptacle device 108 and/or IMD 102 to an anatomical volume of the patient (e.g., the RA). Optionally, delivery catheter 106 may be advanced to the anatomical volume of the patient through a surrounding tubular member (not shown), such as a sheath or guide catheter, which may be placed with its distal end in the anatomical volume before delivery catheter 106 is advanced through the surrounding tubular member. In examples, delivery catheter 106 is configured to retrieve receptacle device 108 and/or IMD 102 from the anatomical volume of the patient. Delivery catheter 106 may include a distal portion 114 (“delivery catheter distal portion 114”) configured to be intracorporeal to the patient and a proximal portion 116 (“delivery catheter proximal portion 116”) which may be extracorporeal to the patient when delivery catheter distal portion 114 is intracorporeal. In examples, delivery catheter distal portion 114 supports (e.g., is attached to and/or is a substantially unitary component with) receptacle device 108. In examples, delivery catheter 106 is configured to deliver and/or retrieve receptacle device 108 and/or IMD 102 using vasculature of a patient, such as an SVC or other vasculature leading to the anatomical volume. In examples, delivery catheter 106 defines a lumen 118 (“delivery lumen 118”) which opens to receptacle volume 112.

In examples, medical system 100 includes a delivery system 120 configured to engage IMD 102 when, for example, IMD 102 is positioned within receptacle volume 112. Delivery system 120 may include a driver 122 that includes a distal portion 124 (“driver distal portion 124”) configured to be intracorporeal to the patient and a proximal portion 126 (“driver proximal portion 126”) which may be extracorporeal to the patient when driver distal portion 124 is intracorporeal. In examples, delivery system 120 includes a head portion 128 supported by driver distal portion 124 and configured to engage IMD 102. At least driver 122 may be configured to slidably translate and/or rotate within delivery lumen 118 (e.g., translate and/or rotate relative to delivery catheter 106). Delivery system 120 may be configured such that the translation and/or rotation of driver 122 causes driver 122 and/or head portion 128 to impart translational and/or rotational forces on IMD 102, such that IMD 102 translates and/or rotates relative to receptacle wall 110. For example, delivery system 120 may be configured to translate within delivery lumen 118 to impart (e.g., via head portion 128) a translational force on IMD 102 causing IMD 102 to translate (e.g., within receptacle volume 112) in a proximal direction P or a distal direction D relative to receptacle wall 110. Delivery system 120 may be configured to rotate within delivery lumen 118 to impart (e.g., via head portion 128) a rotational torque on IMD 102 causing IMD 102 to rotate (e.g., within receptacle volume 112) about a longitudinal axis LD defined by IMD 102. In some examples, head portion 128 and driver 122 may be substantially separate components. In some examples, head portion 128 may be substantially contiguous with driver 122, such that head portion 128 and driver 122 define a unified component. In FIG. 1, portions of driver 122 and/or head portion 128 positioned within delivery lumen 118 and/or receptacle volume 112 are depicted with dashed lines.

A housing 141 of IMD 102 (“IMD housing 141”) includes a distal portion 142 (“housing distal portion 142”) defining a distal surface 138 (“IMD distal surface 138”). IMD distal surface may substantially surround and/or be intersected by longitudinal axis LD. In examples, IMD distal surface 138 includes one or more points defining a distal-most location of IMD housing 141. IMD 102 further includes an attachment member 132 configured to engage tissues within target site 104. Attachment member 132 is configured to extend distally (e.g., in distal direction D) from IMD distal surface 138 to assist in implantation of the IMD and/or assist in maintaining contact between electrodes of IMD 102 and tissues within target site 104. Attachment member 132 may be configured to secure IMD 102 to tissues of heart 101. In examples, IMD housing 141 mechanically supports attachment member 132. Attachment member 132 is configured to penetrate tissue of heart 101 at or near a target site, such as target site 104. For example, attachment member 132 may be configured to penetrate cardiac tissue of a septal wall in a RV, RA, LV, and/or LA of heart 101, or penetrate cardiac tissue in another area of heart 101. Attachment member 132 may be configured to substantially maintain IMD 102 at or in the vicinity of the target site when attachment member 132 penetrates tissues at or in the vicinity of the target site.

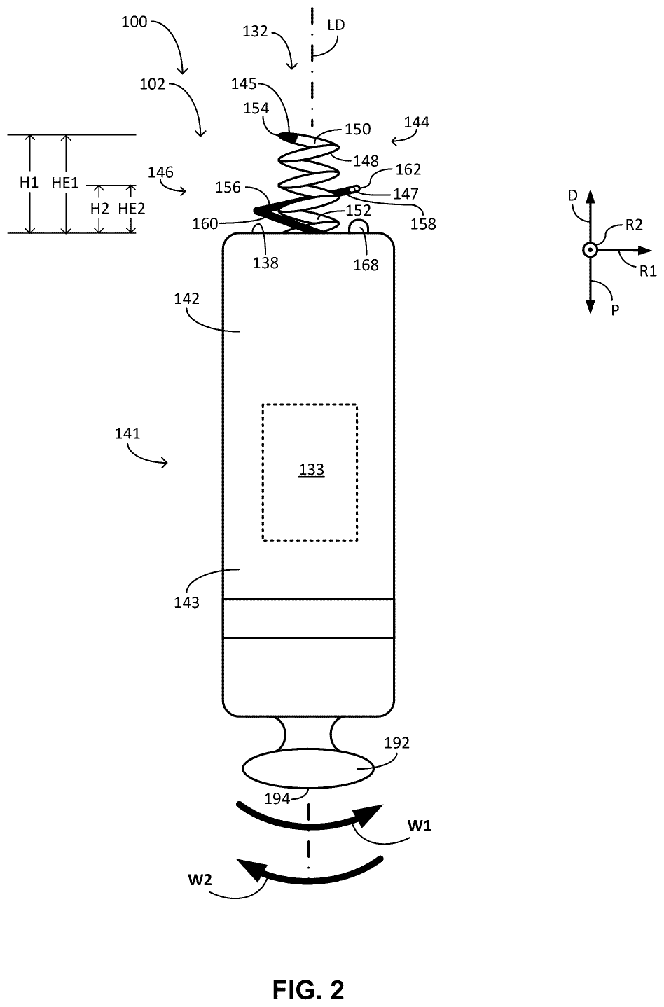

Attachment member 132 includes a primary helix (e.g., primary helix 144 (FIG. 2)) defining a distal end of the primary helix (e.g., primary helix distal end 154 (FIG. 2)), and defines a secondary helix (e.g., secondary helix 146 (FIG. 2) defining a distal end of the secondary helix (e.g., secondary helix distal end 162 (FIG. 2)). In examples, the primary helix is configured such that the primary helix distal end extends distal to housing distal portion 142 and/or IMD distal surface 138. The secondary helix may be configured such that the secondary helix distal end extends distal to housing distal portion 142 and/or IMD distal surface 138. In examples, the primary helix and/or the secondary helix are configured such that the primary helix distal end extends distal to the secondary helix distal end.

The primary helix is configured to displace the primary helix distal end within a tissue wall (e.g., at target site 104) when IMD housing 141 is rotated in one of a first rotary direction W1 or a second rotary direction W2. The secondary helix is configured to displace the secondary distal end within the tissue wall when IMD housing 141 is rotated in the other of the first rotary direction W1 or second rotary direction W2. For example, in some examples, the primary helix defines a right-handed helix in a spatial coordinate system and the secondary helix defines a left-handed helix in the spatial coordinate system. In other examples, the primary helix defines a left-handed helix in the spatial coordinate system and the secondary helix defines a right-handed helix in the spatial coordinate system. Hence, attachment member 132 may be configured such that a rotation of IMD housing 141 in a first direction (e.g., first rotary direction W1) causes the primary helix to embed within the tissue wall, and a rotation of IMD housing 141 in a second direction (e.g., second rotary direction W2) causes the secondary helix to embed within the tissue wall.

In examples, the primary helix supports a first electrode (e.g., first electrode 145 (FIG. 2)) and/or the secondary helix supports a second electrode (e.g., second electrode 147 (FIG. 2)). IMD 102 may include processing circuitry 133 configured to deliver therapy to and/or sense signals from a patient using the first electrode and/or the second electrode. In examples, the primary helix is configured to displace the first electrode within the tissue wall when IMD housing 141 is rotated in one of first rotary direction W1 or second rotary direction W2. The secondary helix may be configured to displace the second electrode within the tissue wall when IMD housing 141 is rotated in the other of first rotary direction W1 or second rotary direction W2. The primary helix and secondary helix may be configured such that a distance in which the first electrode and/or second electrode is embedded within the tissue wall is substantially proportional to an amount and direction of rotation of IMD housing 141. Hence, a clinician may control a depth a insertion of the first electrode and/or the second electrode based on a direction of rotation of IMD housing 141. The clinician may control the depth of insertion using delivery system 120 (e.g., driver 122 and/or head portion 128) to impart a torque to IMD housing 141 in one of the first rotary direction W1 or second rotary direction W2.

Thus, for example, the clinician may impart a torque to IMD housing 1141 in first rotary direction W1 or second rotary direction W2 to cause the primary helix to position the first electrode substantially at a location within the tissue wall sufficient to deliver a first pacing signal to, for example, a conduction system of heart 101 or another portion of heart 101. The clinician may (e.g., subsequently) impart a torque to IMD housing 141 in the other of the first rotary direction W1 or second rotary direction W2 to cause the secondary helix to position the second electrode substantially at a location within the tissue wall sufficient to deliver a second pacing signal to, for example, atrial tissue of heart 101 or another portion of heart 101. The secondary helix (e.g., including the secondary helix distal end) may be configured to slidably translate (e.g., to substantially drag) over a surface of the tissue wall as the clinician imparts torque to IMD housing 141 in the direction causing the primary helix to embed the first electrode until the first electrode is positioned at a suitable location within the tissue wall. The clinician may then impart torque to IMD housing 141 in an opposite direction to cause the secondary helix to embed the second at a suitable location within the tissue wall. Hence, the primary helix and the secondary helix may enable control of a depth of insertion of both the first electrode and the second electrode within the tissue wall, such as a tissue wall of heart 101.

In examples, delivery system 120 (e.g., head portion 128) is configured to engage IMD 102 to transfer a torque to IMD 102. Driver 122 may be configured to receive a torque (e.g., from a clinician) and transfer the torque to head portion 128. Delivery system 120 may be configured to engage with IMD 102 to, for example, implant IMD 102 within an anatomical volume, retrieve IMD 102 from an anatomical volume, re-position IMD 102 within an anatomical volume, and/or re-orient IMD 102 within an anatomical volume. In examples, IMD 102 includes one or more components (e.g., a communication antenna, a sensor, or another component) configured to rotate around and or revolve about longitudinal axis LD when IMD 102 (e.g., IMD housing 141) rotates about longitudinal axis LD. In some examples, instead of or in addition to the first electrode and/or the second electrode, IMD 102 (e.g., IMD distal surface 138) supports an electrode (e.g., an atrial electrode). The first electrode, the second electrode, and/or other electrodes of IMD 102 may be configured to establish electrical communication with tissues and/or other anatomical structures within heart 101 (e.g., tissues and/or other anatomical structures within target site 104).

Medical system 100 may be configured to position IMD 102 in proximity to target site 104 such that IMD 102 may be anchored to tissues within target site 104 (e.g., using attachment member 132). For example, delivery catheter 106 may be configured (e.g., under the influence of a clinician) to traverse vasculature of the patient to position receptacle device 108 and IMD 102 in proximity to target site 104. Medical system 100 (e.g., driver 122 and/or head portion 128) may be configured to impart a torque to IMD 102 to cause attachment member 132 to engage tissues (e.g., tissue within target site 104) when attachment member 132 is within or in proximity to target site 104. In examples, medical system 100 (e.g., driver 122 and/or head portion 128) is configured to impart a force (e.g., in the distal direction D) to IMD 102 to cause attachment member 132 to engage tissues (e.g., tissue within target site 104) when attachment member 132 is within or in proximity to target site 104. Medical system 100 may be configured such that driver 122 and/or head portion 128 may be disengaged from IMD 102 as IMD 102 remains anchored to tissues within or in proximity to target site 104. Delivery catheter 106, receptacle device 108, and delivery system 120 may subsequently be withdrawn from the patient (e.g., via vasculature of the patient).

In examples, medical system 100 is configured to deliver therapy to a patient and/or sense physiological signals of the patient received when receptacle device 108 is positioned (e.g., by a clinician) within heart 101. In some examples, medical system 100 may be configured to sense an indication of an intrinsic cardiac electrical signal produced by heart 101. Medical system 100 may be configured to process and/or condition a sensed signal to provide, for example, an indication of a location of receptacle device 108 and/or IMD 102 within heart 101, to conduct pace mapping for deployment of IMD 102, to provide indications indicative of the deployment and/or attachment of IMD 102 within heart 101, or for other reasons. In examples, medical system 100 includes processing circuitry 134 configured to deliver therapy and/or sense physiological signals. In examples, processing circuitry 134 includes processing circuitry such as device processing circuitry 131, configured to be mechanically supported by an external device 136 and/or another device of medical system 100. External device 136 and/or device processing circuitry 131 may be configured to be extracorporeal to the patient and/or otherwise displaced from receptacle device 108 and/or IMD 102 when, for example, receptacle device 108 and/or IMD 102 are intracorporeal to the patient. In examples, processing circuitry 134 includes processing circuitry such as IMD processing circuitry 133, configured to be mechanically supported by IMD 102 (e.g., IMD housing 141) and/or another device of medical system 100. IMD processing circuitry 133 may be configured to be intracorporeal to the patient when, for example, receptacle device 108 and/or IMD 102 are intracorporeal to the patient. Processing circuitry 134 may be configured to deliver therapy and/or sense physiological signals using one or more electrodes of medical system 100, such as the first electrode, the second electrode, another electrode supported by IMD 102, one or more electrodes supported by receptacle device 108, one or more electrodes supported by delivery system 120, an external electrode 139 configured to be extracorporeal to the patient, and/or one or more other electrodes in communication with and/or supported by medical system 100. In examples, processing circuitry 134 is configured to communicate with one or more electrodes using one or more communication links, such as communication link 135, communication link 137, and/or other communication links. In some examples, medical system 100 may include a snare configured to engage at least some portion of IMD 102 (e.g., IMD retrieval structure 192 (FIG. 2)).

Although the examples herein discuss delivery, retrieval, and/or positioning of IMD 102 within the RA of heart 101, medical system 100 may be configured to deliver, retrieve, and/or position IMD 102 in any of the other chambers of heart 101 and/or in other anatomical volumes of a patient in a like manner as that described for the RA of heart 101. Further, although the examples herein discuss attachment member 132 defining a primary helix and a secondary helix, attachment member 132 may define other structures, such as one or more elongated tines extending from, for example, housing distal portion 142. Further, delivery system 120 may be configured to exert a translational force (e.g., in the distal direction D) on IMD 102 to, for example, cause and/or assist attachment member 132 in engaging tissues. Delivery system 120 may be configured to exert a translational force (e.g., in the proximal direction P) on IMD 102 to, for example, cause and/or assist attachment member 132 in disengaging from tissues. The translational force exerted by delivery system 120 may cause IMD 102 to move in the distal direction D or the proximal direction P relative to receptacle device 108, heart 101, and/or other portions of medical system 100. Target site 104 may include an appendage of the RA, a triangle of Koch region of the RA, some other portion of heart 101, or some other location within a body of a patient.

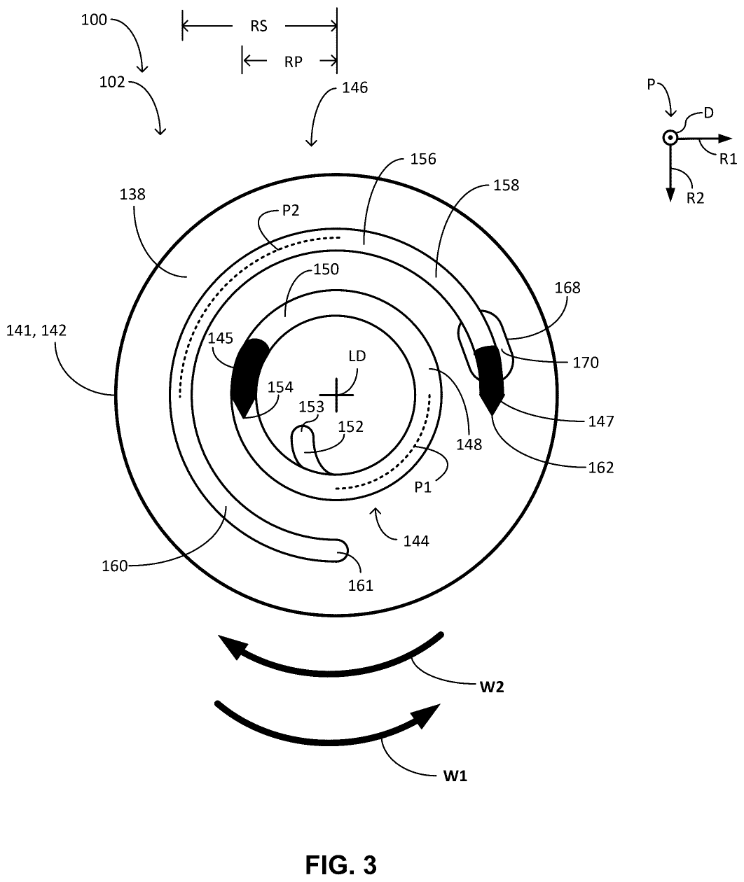

FIG. 2 is a schematic plan view of IMD 102 including a primary helix 144 and a secondary helix 146, with a distal direction D and a proximal direction P parallel to the page. IMD housing 141 defines housing distal portion 142 and a proximal portion 143 (“housing proximal portion 143”) proximal to housing distal portion 142. IMD 102 defines a longitudinal axis LD extending through at least housing distal portion 142 and housing proximal portion 143. In examples, distal direction D and proximal direction P are substantially parallel to longitudinal axis L. IMD 102 further defines a first radial direction R1 substantially perpendicular to longitudinal axis LD and defines a second radial direction R2 substantially perpendicular to longitudinal axis L and substantially perpendicular to first radial direction R1. In examples, distal direction D, first radial direction R1, and second radial direction R2 define a right-hand coordinate system (“R1-D-R2 coordinate system”) which rotates and/or translates synchronously with IMD housing 141 about and/or over one or more common axes. FIG. 3 is schematic end view of IMD 102 including primary helix 144 and secondary helix 146, with distal direction D proceeding out of the page and first radial direction R1 and second radial direction R2 parallel to the page.

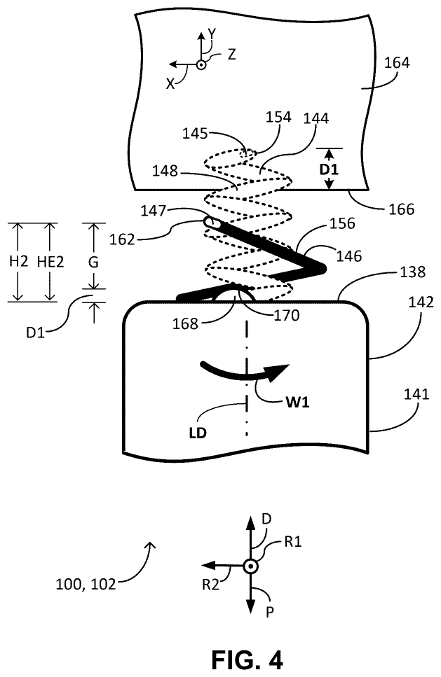

In FIG. 2, FIG. 3, and FIGS. 4-8B, primary helix 144 is depicted as a helical body defining a helical path (e.g., helical path P1 (FIG. 3)) configured to advance distally when viewed along longitudinal axis L in distal direction D and rotated clockwise about longitudinal axis L (e.g., a right-handed helix). Secondary helix 146 is depicted as a helical body defining a helical path (e.g., helical path P2 (FIG. 3)) configured to advance distally when viewed along longitudinal axis L in distal direction D and rotated counter-clockwise about longitudinal axis L (e.g., a left-handed helix). However, it is understood that these specific orientations of primary helix 144 and secondary helix 146 are not required and that the depictions and subsequent discussion are examples used consistently for clarity of illustration and discussion. For example, in some examples, primary helix 144 may be a left-handed helix and secondary helix 146 may be a right-handed helix. In some examples, the “first rotational direction” discussed herein is one of first rotary direction W1 or second rotary direction W2 and the “second rotational direction” discussed herein is the other of first rotary direction W1 or second rotary direction W2.

IMD 102 is configured such that primary helix 144 tends to embed with a tissue wall when primary helix 144 is rotated in a first rotational direction (e.g., one of first rotary direction W1 or second rotary direction W2). IMD 102 is configured such that secondary helix 146 tends to embed with the tissue wall when secondary helix 146 is rotated in a second rotational direction opposite the first rotational direction (e.g., the other of first rotary direction W1 or second rotary direction W2). In examples, primary helix 144 supports a first electrode 145 and/or secondary helix 146 supports a second electrode 147. IMD 102 is configured such that an implantation of first electrode 145 and/or second electrode 147 may be controlled based on a direction and/or amount of rotation of IMD housing 141.

For example, a clinician may impart a torque to IMD housing 141 in the first rotational direction to cause primary helix 144 to position first electrode 145 at a first location within a tissue wall. The first position may be, for example, a position sufficient to deliver a first pacing signal to a first portion of heart 101 (FIG. 1). The clinician may (e.g., subsequently) impart a torque to IMD housing 141 in the second rotational direction opposite the first direction to cause secondary helix 146 to position second electrode 147 at a second location within the tissue wall. The second rotational location may be, for example, sufficient to deliver a second pacing signal to a second portion of heart 101. Hence, IMD 102 is configured such that the clinician may control the positioning of first electrode 145 and second electrode 147 within the tissue wall based on a direction of rotation of IMD housing 141.

Primary helix 144 may be configured such that a depth of insertion of first electrode 145 is dependent on an amount of rotation of IMD housing 141. In examples, primary helix 144 is configured to increase a depth of first electrode 145 relative to a tissue surface of the tissue wall as primary helix 144 rotates in the first rotational direction. For example, primary helix 144 may be configured such that a first degree of rotation of primary helix 144 in the first rotational direction causes first electrode 145 to embed within the tissue wall to a first depth. Primary helix 144 may be configured such that a second degree of rotation in the first rotational direction greater than the first degree of rotation causes first electrode 145 to embed within the tissue wall to a second depth greater than the first depth.

In contrast, primary helix 144 may be configured to decrease a depth of first electrode 145 relative to the tissue surface as primary helix 144 rotates in the second rotational direction opposite the first direction. For example, primary helix 144 may be configured such that a degree of rotation of primary helix 144 in the second rotational direction causes first electrode 145 to decrease its depth from the second depth to another depth less than the second depth. Hence, IMD 102 may be configured to allow control of the depth of first electrode 145 through control of a direction and degree of rotation of primary helix 144. In examples, IMD 102 is configured such that a rotation of IMD housing 141 about longitudinal axis L causes a rotation of primary helix 144 in the same rotational direction about longitudinal axis L such that, for example, IMD 102 allows control of the depth of first electrode 145 using a direction and degree of rotation of IMD housing 141.

Secondary helix 146 may be configured such that a depth of insertion of second electrode 147 is dependent on an amount of rotation of IMD housing 141. In examples, secondary helix 146 is configured to increase a depth of second electrode 147 relative to the tissue surface of the tissue wall as secondary helix 146 rotates in the second rotational direction opposite the first direction. For example, secondary helix 146 may be configured such that a third degree of rotation of secondary helix 146 in the second rotational direction causes second electrode 147 to embed within the tissue wall to a third depth. Secondary helix 146 may be configured such that a fourth degree of rotation in the second rotational direction greater than the third degree of rotation causes second electrode 147 to embed within the tissue wall to a fourth depth greater than the third depth.

In contrast, secondary helix 146 may be configured to decrease a depth of second electrode 147 relative to the tissue surface as secondary helix 146 rotates in the first rotational direction opposite the second rotational direction. For example, secondary helix 146 may be configured such that a degree of rotation of secondary helix 146 in the first rotational direction causes second electrode 147 to decrease its depth from the fourth depth to another depth less than the fourth depth. Hence, IMD 102 may be configured to allow control of the depth of second electrode 147 through control of a direction and degree of rotation of secondary helix 146. In examples, IMD 102 is configured such that a rotation of IMD housing 141 about longitudinal axis L causes a rotation of secondary helix 146 in the same rotational direction about longitudinal axis L such that, for example, IMD 102 allows control of the depth of second electrode 147 using a direction and degree of rotation of IMD housing 141.

Hence, primary helix and the secondary helix may enable control of a depth of insertion of both the first electrode and the second electrode within the tissue wall, such as a tissue wall of heart 101. Thus, IMD 102 is configured such that a clinician may impart a torque to IMD housing 141 in the first rotational direction (e.g., first rotary direction W1 or second rotary direction W2) to cause primary helix 144 to position first electrode 145 substantially at a location within a tissue wall sufficient to deliver a first pacing signal to, for example, a conduction system of heart 101 or another portion of heart 101. IMD 102 is configured such that the clinician may (e.g., subsequently) impart a torque to IMD housing 141 in the second rotary direction (e.g., the other of the first rotary direction W1 or second rotary direction W2) to cause secondary helix 146 to position second electrode 147 substantially at a location within the tissue wall sufficient to deliver a second pacing signal to, for example, atrial tissue of heart 101 or another portion of heart 101.

Primary helix 144 includes a helical body 148 (“primary helical body 148”) defining a distal portion 150 (“primary helix distal portion 150”) and a proximal portion 152 (“primary helix proximal portion 152”). Primary helix distal portion 150 defines a distal end 154 of primary helical body 148 (“primary helix distal end 154”). In examples, primary helix proximal portion 150 extends from and/or is supported by housing distal portion 142. Primary helix 144 (e.g., primary helical body 148) is configured to extend in distal direction D beyond housing distal portion 142. For example, IMD 102 may be configured such that, when primary helix proximal portion 152 extends from and/or is supported by housing distal portion 142, primary helix distal portion 150 is displaced in distal direction D from IMD distal surface 138. IMD 102 may be configured such that, when primary helix proximal portion 152 extends from and/or is supported by housing distal portion 142, primary helix distal end 154 is displaced in distal direction D from IMD distal surface 138.

Primary helical body 148 may support first electrode 145. In examples, primary helix distal portion 150 supports first electrode 145. In some examples, primary helix distal portion 150 supports first electrode 145 at or in proximity to primary helix distal end 154. In some examples, first electrode 145 is mechanically supported by primary helical body 148. In some examples, first electrode 145 comprises some portion of a surface of primary helical body 148. For example, primary helical body 148 may include a conductive material covered at least in part by an insulative material. First electrode 145 may be a surface defined by some portion of the conductive material having the insulative cover removed.

In examples, IMD 102 is configured such that primary helix 144 defines a first helical height H1 from housing distal portion 142 (e.g., IMD distal surface 138) to primary helix distal end 154 (e.g., when primary helix proximal portion 152 extends from and/or is supported by housing distal portion 142). First helical height H1 may be substantially parallel to longitudinal axis L. IMD 102 may be configured such a rotation of IMD housing 141 about longitudinal axis L causes a rotation of primary helix 144 in the same direction about longitudinal axis L. In examples, primary helix 144 substantially surrounds longitudinal axis L.

In some examples, IMD 102 is configured such that primary helix 144 defines a first electrode height HE1 from housing distal portion 142 (e.g., IMD distal surface 138) to first electrode 145 (e.g., to one or more points on a surface of first electrode 145). Primary helix 144 may define first electrode height HE1 when primary helix proximal portion 152 extends from and/or is supported by housing distal portion 142. In examples, first electrode height HE1 is substantially parallel to longitudinal axis L and/or first helical height H1. In some examples, IMD 102 is configured such that first electrode height HE1 is substantially equal to first helical height H1. In some examples, IMD 102 is configured such that first electrode height HE1 is less than first helical height H1. Primary helical body 148 may support first electrode 145 on any portion of primary helix distal portion 150 and/or primary helix proximal portion 152.

In examples, primary helical body 148 defines a first helical path P1 around longitudinal axis LD. First helical path P1 may extend substantially from housing distal portion 142 (e.g. IMD distal surface 138) to primary helix distal end 154. In examples, first helical path P1 defines a first helical length between housing distal portion 142 (e.g. IMD distal surface 138) and primary helix distal end 154, and defines a midpoint of first helical path P1 substantially halfway between housing distal portion 142 and primary helix distal end 154. Primary helix distal portion 150 may extend from the midpoint of first helical path P1 to primary helix distal end 154. Primary helix proximal portion 152 may substantially extend from housing distal portion 142 (e.g. IMD distal surface 138) to the midpoint of first helical path P1. In examples, primary helix proximal portion 152 extends from an intersection of primary helical body 148 and a plane tangent to IMD distal surface 138 and substantially perpendicular to longitudinal axis LD. In examples, primary helix proximal portion 152 extends from a base portion 153 (“primary helix base portion 153”) supported by housing distal portion 142 and/or located at the intersection of primary helical body 148 and the plane tangent to IMD distal surface 138.

Secondary helix 146 includes a helical body 156 (“secondary helical body 156”) defining a distal portion 158 (“secondary helix distal portion 158”) and a proximal portion 160 (“secondary helix proximal portion 160”). Secondary helix distal portion 158 defines a distal end 162 of secondary helical body 156 (“secondary helix distal end 162”). In examples, secondary helix proximal portion 160 extends from and/or is supported by housing distal portion 142. Secondary helix 146 (e.g., secondary helical body 156) is configured to extend in distal direction D beyond housing distal portion 142. For example, IMD 102 may be configured such that, when secondary helix proximal portion 160 extends from and/or is supported by housing distal portion 142, secondary helix distal portion 158 is displaced in distal direction D from IMD distal surface 138. IMD 102 may be configured such that, when secondary helix proximal portion 160 extends from and/or is supported by housing distal portion 142, secondary helix distal end 162 is displaced in distal direction D from IMD distal surface 138.

Secondary helical body 156 may support second electrode 147. In examples, secondary helix distal portion 158 supports second electrode 147. In some examples, secondary helix distal portion 158 supports second electrode 147 at or in proximity to secondary helix distal end 162. In some examples, second electrode 147 is mechanically supported by secondary helical body 156. In some examples, second electrode 147 comprises some portion of a surface of secondary helical body 156. For example, secondary helix body 148 may include a second conductive material covered at least in part by an insulative layer. Second electrode 147 may be a surface defined by some portion of the second conductive material having the insulative layer removed.

In examples, IMD 102 is configured such that secondary helix 146 defines a second helical height H2 from housing distal portion 142 (e.g., IMD distal surface 138) to secondary helix distal end 162 (e.g., when secondary helix proximal portion 160 extends from and/or is supported by housing distal portion 142). Second helical height H2 may be substantially parallel to longitudinal axis L and/or first helical height H1. IMD 102 may be configured such a rotation of IMD housing 141 about longitudinal axis L causes a rotation of secondary helix 146 in the same direction about longitudinal axis L. In examples, secondary helix 146 substantially surrounds longitudinal axis L.

In some examples, IMD 102 is configured such that secondary helix 146 defines a second electrode height HE2 from housing distal portion 142 (e.g., IMD distal surface 138) to second electrode 147 (e.g., to one or more points on a surface of second electrode 147). Secondary helix 146 may define second electrode height HE2 when secondary helix proximal portion 160 extends from and/or is supported by housing distal portion 142. In examples, second electrode height HE2 is substantially parallel to longitudinal axis L, first helical height H1, first electrode height HE1, and/or second helical height H2. In some examples, IMD 102 is configured such that second electrode height HE2 is substantially equal to second helical height H2. In some examples, IMD 102 is configured such that second electrode height HE2 is less than second helical height H2. Secondary helical body 156 may support second electrode 147 on any portion of secondary helix distal portion 158 and/or secondary helix proximal portion 160.

The relative displacements defined among first helical height H1, first electrode height HE1, second helical height H2, and/or second electrode height HE2 may, in some examples, be based on relative positions of first electrode 145 and second electrode 147 desired within a tissue wall when primary helix 144 and secondary helix 146 are implanted within the tissue wall. For example, the relative displacements may be based on a desire to stimulate and/or sense tissue at a first depth within the tissue wall using first electrode 145 while stimulating and/or sensing tissue at a second depth within the tissue wall using second electrode 147. The relative displacements defined among first helical height H1, first electrode height HE1, second helical height H2, and/or second electrode height HE2 may be based on an expected displacement between the first depth and the second depth. For example, the first depth may be indicative of a depth at which first electrode 145 could be expected to stimulate a conduction system of heart 101 (FIG. 1). The second depth might be indicative of a depth at which second electrode 145 could be expected to stimulate atrial tissue of heart 101.

In examples, IMD 102 is configured such that primary helix 144 extends distal to secondary helix 146. For example, IMD 102 (e.g., primary helix 144 and secondary helix 146) may be configured such that first helical height H1 is greater than second helical height H2. In some examples, IMD 102 is configured such that second helical height H2 is less than about 50%, in some examples less than about 25%, of first helical height H1. IMD 102 (e.g., primary helix 144 and secondary helix 146) may be configured such that first electrode height HE1 is greater than second electrode height H2. For example, IMD 102 may be is configured such that second electrode height H2 is less than about 50%, in some examples less than about 25%, of first electrode height H1.

In examples, secondary helical body 156 defines a second helical path P2 around longitudinal axis LD. Second helical path P2 may extend substantially from housing distal portion 142 (e.g. IMD distal surface 138) to secondary helix distal end 162. In examples, second helical path P2 defines a first helical length between housing distal portion 142 (e.g. IMD distal surface 138) and secondary helix distal end 162, and defines a midpoint of second helical path P2 substantially halfway between housing distal portion 142 and secondary helix distal end 162. Secondary helix distal portion 158 may extend from the midpoint of second helical path P2 to secondary helix distal end 162. Secondary helix proximal portion 160 may substantially extend from housing distal portion 142 (e.g. IMD distal surface 138) to the midpoint of second helical path P2. In examples, secondary helix proximal portion 160 extends from an intersection of secondary helical body 156 and a plane tangent to IMD distal surface 138 and substantially perpendicular to longitudinal axis LD. In examples, secondary helix proximal portion 160 extends from a base portion 161 (“secondary helix base portion 161”) supported by housing distal portion 142 and/or located at the intersection of secondary helical body 156 and the plane tangent to IMD distal surface 138.

In examples, IMD distal surface 138 includes a substantially planar surface substantially perpendicular to longitudinal axis LD, although this is not required. IMD distal surface 138 may include a surface defining one or more profiles of profile segments that are substantially curved, curvilinear, or linear. In some examples, IMD distal surface 138 may include a surface of revolution defined by rotating a generatrix about longitudinal axis LD. In examples, IMD distal surface 138 defines a point that defines a distal-most extension of IMD housing 141. For example, IMD distal surface 138 may substantially define a distal end of IMD housing 141. IMD 102 may be configured such that primary helix distal end 154 and secondary helix distal end 162 position distal to the point defining the distal-most extension of IMD housing 141.

In some examples, secondary helix 146 substantially surrounds primary helix 144. For example, primary helix 144 (e.g., primary helical body 148) may define a radial displacement RP from longitudinal axis LD. Secondary helix 146 (e.g., secondary helical body 156) may define a radial displacement RS from longitudinal axis LD. In examples, IMD 102 is configured such that radial displacement RS is greater than radial displacement RP. In some examples, primary helix 144 substantially surrounds secondary helix 146. For example, IMD 102 may be configured such that radial displacement RP is greater than radial displacement RS. In some examples, radial displacement RP is substantially equal to radial displacement RS. Radial displacement RP and/or radial displacement RS may be substantially perpendicular to longitudinal axis LD. In examples, radial displacement RP is substantially parallel to radial displacement RS. In some examples, primary helix 144 extends substantially between longitudinal axis L and secondary helix 146. In some examples, secondary helix 146 extends substantially between longitudinal axis L and primary helix 144.

As used herein, a helix and/or helical body may refer to a body defining a corkscrew-type shape around an axis such as longitudinal axis LD. The corkscrew-type shape may substantially spiral around an axis (e.g., a linear axis). For example, primary helical body 148 may define a first corkscrew-type shape substantially spiraling around longitudinal axis LD or another axis and extending from primary helix proximal portion 152 to about primary helix distal end 154. Secondary helical body 156 may defines a second corkscrew-type shape substantially spiraling around longitudinal axis LD or another axis and extending from secondary helix proximal portion 160 to about secondary helix distal end 162. In some examples, primary helical body 148 defines a first substantially circular helix (e.g., about longitudinal axis LD or another axis) and/or secondary helical body 156 defines a second substantially circular helix (e.g., about longitudinal axis LD or another axis), although this is not required. Primary helical body 148 and/or secondary helical body 156 may define a conic helix, a general helix, a cylindrical helix, a slant helix, a conic helix, a space spiral, or other like shapes which substantially spiral around an axis.

As used herein, when a helix and/or helical body substantially surrounds an axis or a component (e.g., such as a second helix or helical body), in some examples, this may refer to the helix and/or helical body defining a path which curves around at least some portion of the axis or the component and defines a linear translation with respect to the axis or the component. In some examples, the path completes at least one complete turn around the portion of the axis or the component. In some examples, the path completes less than one complete turn around the portion of the axis or the component. For example, when secondary helix 146 substantially surrounds primary helix 144 and/or longitudinal axis LD, this may mean secondary helix 146 defines a path that completes less than one complete turn around primary helix 144 and/or longitudinal axis LD in some examples, or may mean secondary helix 146 defines a path which completes at least one complete turn around primary helix 144 and/or longitudinal axis LD. When primary helix 144 substantially surrounds secondary helix 146 and/or longitudinal axis LD, this may mean primary helix 144 defines a path that completes less than one complete turn around secondary helix 146 and/or longitudinal axis LD in some examples, or may mean primary helix 144 defines a path which completes at least one complete turn around secondary helix 146 and/or longitudinal axis LD in other examples.

As discussed, primary helix 144 may be configured such that a rotation of IMD housing 141 in a first rotational direction (e.g., first rotary direction W1) causes primary helix 144 to embed primary helix distal end 154 within a tissue wall. Secondary helix 146 may be configured to contact the tissue wall when primary helix 144 rotates in the first rotational direction to embed primary helix distal end 154 within the tissue wall. In examples, secondary helix 146 (e.g., including secondary helix distal end 162) is configured to slidably translate (e.g., to substantially drag) over the surface of the tissue wall when primary helix 144 rotates in the first rotational direction to embed (or, e.g., further embed) primary helix distal end 154 within the tissue wall. Secondary helix 146 may be configured to slidably translate over the surface of the tissue wall when secondary helix 146 (e.g., secondary helix distal end 162) is in contact with the tissue wall. In some examples, secondary helix 146 is configured to substantially compress toward IMD housing 141 (e.g., toward IMD distal surface 138) when secondary helix 146 contacts the tissue wall as primary helix 144 rotates to embed primary helix distal end 154 within the tissue wall.

Secondary helix 146 is configured to embed secondary helix distal end 162 within the tissue wall when IMD housing 141 is rotated in a second rotational direction opposite the first rotational direction (e.g., second rotary direction W2). IMD 102 may be configured to cause secondary helix 146 to embed secondary helix distal end 162 within the tissue wall when IMD housing 141 is rotated in the second rotational direction. For example, IMD 102 may be configured to cause secondary helix distal end 162 to remain distally displaced from IMD distal surface 138 and/or another portion of IMD housing 141, such that secondary helix distal end 162 tends to penetrate the tissue wall when IMD housing 141 is rotated in the second rotational direction. Stated similarly, IMD 102 may be configured to limit and/or substantially minimize a tendency of secondary helix distal end 162 to establish a position substantially flush against IMD distal surface 138. Such a substantially flush position might otherwise introduce difficulty in causing secondary helix distal end 162 to penetrate the tissue wall using a rotation of IMD housing 141 in the second rotational direction.

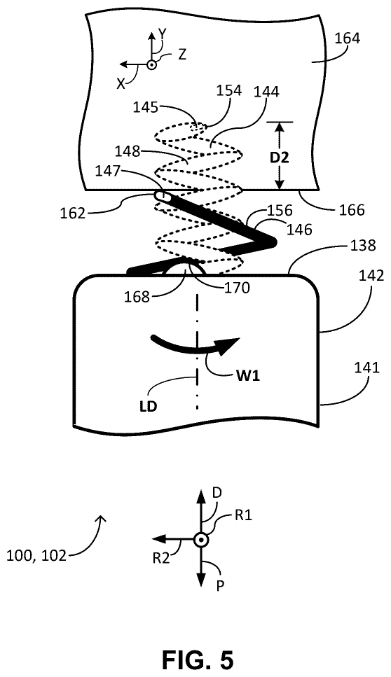

For example, FIG. 4 is a schematic view of IMD 102 engaging a tissue wall 164 of a patient, with IMD housing 141 having experienced rotation in first rotary direction W1 sufficient to cause primary helix distal end 154 and/or first electrode 145 to embed to a depth of about depth D1 relative to a tissue wall surface 166 of tissue wall 164. In FIG. 4, secondary helix 146 is proximally displaced from tissue wall surface 166. FIG. 5 is a schematic view of IMD 102 having further rotated in first rotary direction W1 to increase the depth of primary helix distal end 154 and/or first electrode 145 from depth D1 to a depth of about depth D2. In FIG. 5, secondary helix 146 (e.g., secondary helix distal end 163 and/or second electrode 147) is in contact with tissue wall surface 166.

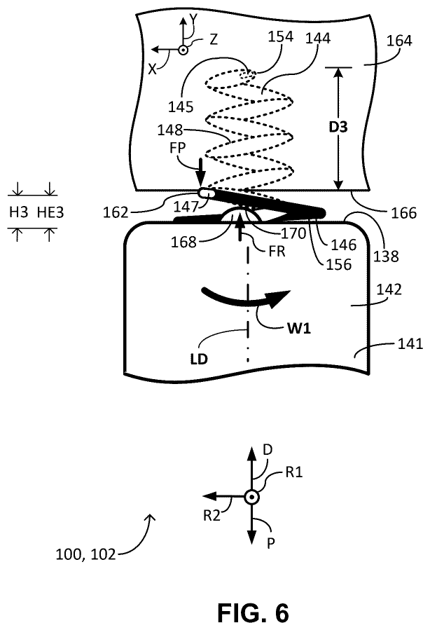

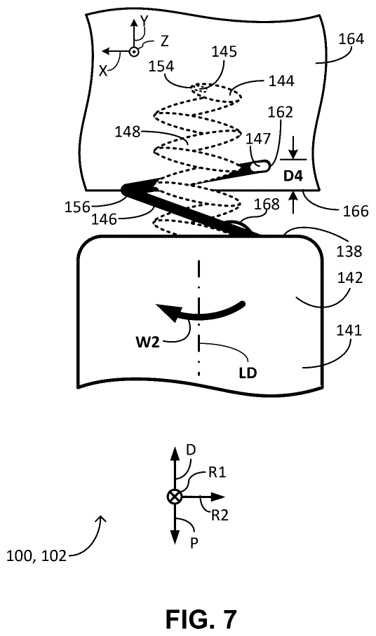

FIG. 6 is a schematic view of IMD 102 having further rotated in first rotary direction W1 to increase the depth of primary helix distal end 154 and/or first electrode 145 from depth D2 to a depth of about depth D3. In FIG. 6, secondary helix 146 (e.g., secondary helix distal end 162 and/or second electrode 147) remains in contact with tissue wall surface 166 (although this is not required). FIG. 7 is a schematic view of IMD 102 having rotated in second rotary direction W2 to cause secondary helix 146 to embed secondary helix distal end 162 and/or second electrode 147 within tissue wall 164 to a depth of about depth D4.

In FIG. 4, FIG. 5, FIG. 6, and FIG. 7, primary helix 144 is depicted with dashed lines and secondary helix 146 is depicted with solid lines for clarity. For clarity, FIG. 4, FIG. 5, FIG. 6, and/or FIG. 7 further depict a right-handed coordinate system defined by tissue wall 164 (“X-Y-Z coordinate system”) and including an x-axis, a y-axis substantially perpendicular to the x-axis, and a z-axis substantially perpendicular to the x-axis and the y-axis. In examples, in FIG. 4, FIG. 5, FIG. 6, and/or FIG. 7, rotation of IMD housing 141 in first rotary direction W1 and/or second rotary direction W2 causes rotation of the R1-D-R2 coordinate system defined by IMD 102 relative to the X-Y-Z coordinate system defined by tissue wall 164. For example, FIG. 5 depicts a rotation of IMD 102 relative to tissue wall 164 of about 360 degrees compared to FIG. 4. FIG. 6 depicts a rotation of IMD 102 relative to tissue wall 164 of about 360 degrees compared to FIG. 5. FIG. 7 depicts a rotation of IMD 102 relative to tissue wall 164 of about 180 degrees compared to FIG. 6. However, these specific rotations are depicted for clarity of explanation and are not specifically required for the operations of IMD 102 discussed with reference to FIG. 4, FIG. 5, FIG. 6, FIG. 7, and other figures of this disclosure.

Primary helix distal end 154 may be configured to penetrate tissue wall surface 166 (e.g., when IMD housing 141 is rotated in first rotary direction W1). Primary helix 144 (e.g., primary helical body 148) may be configured to substantially tunnel into tissue wall 164 in a substantially helical path (e.g., around longitudinal axis LD) when IMD housing 141 is rotated in first rotary direction W1. Primary helical body 148 may be configured such that primary helix distal end 154 traverses the substantially helical path as primary helix 144 substantially tunnels into tissue wall 164. Primary helical body 148 may be configured such that primary helix distal end 154 translates in distal direction D relative to tissue wall surface 166 as primary helix distal end 154 traverses the substantially helical path. Primary helical body 148 may be configured such that first electrode 145 traverses at least some portion (e.g., substantially all) of the substantially helical path and/or translates in distal direction D relative to tissue wall surface 166 as primary helix 144 substantially tunnels into tissue wall 164. Hence, primary helical body 148 may be configured to increase a depth of first electrode 145 relative to tissue wall surface 166 as IMD housing 141 is rotated in a first rotational direction (e.g., first rotary direction W1).

Primary helix 144 (e.g., primary helical body 148) may be configured to substantially retreat from (e.g., to back out of) tissue wall 164 when IMD housing 141 is rotated in second rotary direction W2. In examples, primary helical body 148 is configured to substantially retreat from tissue wall 164 over the substantially helical path. Primary helical body 148 may be configured such that primary helix distal end 154 and/or first electrode 145 retreats from tissue wall 164 (e.g., over the substantially helical path) as primary helix 144 retreats from tissue wall 164. For example, primary helical body 148 may be configured such that primary helix distal end 154 and/or first electrode 145 translates in proximal direction P relative to tissue wall surface 166 as primary helix distal end 154 retreats from tissue wall 164 (e.g., as primary helix distal end 154 traverses the substantially helical path). Hence, primary helical body 148 may be configured to decrease a depth of first electrode 145 relative to tissue wall surface 166 as IMD housing 141 is rotated in a second rotational direction (e.g., second rotary direction W2).

Thus, IMD 102 may be configured such that a clinician may control a depth of insertion of first electrode 145 relative to tissue wall surface 166 using a direction of rotation and an amount of rotation of IMD housing 141. The clinician may cause IMD housing 141 to rotate in the first rotational direction (e.g., using driver 122 (FIG. 1)) to increase the depth of first electrode 145 relative to tissue wall surface 166. The clinician may cause IMD housing 141 to rotate in the second rotational direction (e.g., using driver 122) to decrease the depth of first electrode 145 relative to tissue wall surface 166. The clinician may conduct mapping and/or other sensing using processing circuitry 134 (FIG. 1) as the clinician increases or decreases the depth of first electrode 145 relative to tissue wall surface 166. In examples, IMD 102 is configured to impart a force in distal direction D on primary helix 144 to assist in causing primary helix distal end 154 to penetrate tissue wall surface 166 and/or cause primary helical body 148 to substantially tunnel into tissue wall 164 in the substantially helical path. For example, IMD housing 141 may be configured to receive a force in distal direction D from driver 122 (e.g., a force imparted by a clinician) and transfer the force in distal direction D to primary helical body 148.

Referring primary to FIG. 5, IMD 102 (e.g., secondary helix 146 and/or primary helix 144) may be configured to cause a distance between secondary helix 146 and tissue wall surface 166 to decrease as primary helix 144 increases a depth of primary helix distal end 154 and/or first electrode 145 (e.g., as primary helix 144 increases the depth from about depth D1 to about depth D2). In examples, IMD 102 (e.g., secondary helix 146 and/or primary helix 144) is be configured to cause a distance between IMD housing 141 (e.g., IMD distal surface 138) and tissue wall surface 166 to decrease as primary helix 144 increases a depth of primary helix distal end 154 and/or first electrode 145. In examples, IMD 102 (e.g., secondary helix 146 and/or primary helix 144) is configured to cause secondary helix distal end 162 to move in distal direction D toward tissue wall surface 166 as primary helix 144 increases a depth of primary helix distal end 154 and/or first electrode 145.

Secondary helix 146 may be configured to substantially slidably translate over tissue wall surface 166 when at least some portion of secondary helix 146 (e.g., secondary helix distal portion 158 and/or secondary helix distal end 162) contacts tissue wall surface 166 and IMD housing 141 rotates in first rotary direction W1. The portion of secondary helix 146 may be configured to slidably translate over tissue wall surface 166 in a substantially atraumatic manner. In examples, secondary helix distal end 162 is configured to substantially drag over tissue wall surface 166 when secondary helix 146 contacts tissue wall surface 166 and IMD housing 141 rotates in first rotary direction W1. For example, secondary helix 146 may be configured such that secondary helix distal end 162 substantially traces a circular path on tissue wall surface 166 (e.g., a circular path having a radius of about radial displacement RS (FIG. 3)) when secondary helix distal end 162 contacts tissue wall surface 166 and IMD housing 141 rotates in first rotary direction W1.

In some examples, for example as depicted in FIG. 6, secondary helix 146 is configured to substantially compress toward IMD housing 141 (e.g., IMD housing distal portion 142) when secondary helix 146 contacts tissue wall surface 166 and IMD 102 causes primary helix distal end 154 and/or first electrode 145 to translate in distal direction D relative to tissue wall surface 166 (e.g., when IMD housing is rotated in first rotary direction W1). Secondary helix 146 may be configured to cause secondary helix distal end 162 to move in proximal direction P relative to IMD housing 141 (e.g., IMD distal surface 138) when secondary helix 146 substantially compresses toward IMD housing 141. For example, IMD 102 may be configured such that secondary helix 146 causes secondary helix distal end 162 to define a third helical height H3 less than helical height H2 (FIG. 2) when secondary helix 146 substantially compresses toward IMD housing 141. IMD 102 may be configured such that secondary helix 146 causes second electrode 147 to define a third electrode height HE3 less than electrode height HE2 (FIG. 2) when secondary helix 146 substantially compresses toward IMD housing 141.

Secondary helix 146 (e.g., secondary helical body 156) may be resiliently biased to cause secondary helix 146 to define second helical height H2 and/or second electrode height HE2. The resilient biasing may result in a tendency of secondary helical body 156 to return or attempt to return to an initial position defined by second helical height H2 and/or second electrode height HE2 when secondary helical body 156 is temporarily displaced (e.g., departs from) from the initial position. Secondary helical body 156 may be configured such that, when a force (e.g., imparted from tissue wall surface 166) is exerted on secondary helical body 156 and causes secondary helix distal end 162 to move in proximal direction P relative to IMD housing 141, a material comprising secondary helical body 156 (e.g., a shape memory material) urges secondary helical body 156 to return or attempt to return to the initial position by causing secondary helical body 156 to exert a reaction force (e.g., on tissue wall surface 166) opposing the exerted force. In examples, secondary helical body 156 is configured to substantially maintain the initial position defined by second helical height H2 and/or second electrode height HE2 when secondary helical body 156 is in a substantially zero-stress position, where any stresses on secondary helical body 156 arise from properties or phenomena internal to secondary helical body 156, such as mass, internal temperature, residual stresses (e.g., from fabrication and/or mechanical attachment to another component), and the like.

Referring to FIG. 7, secondary helix distal end 162 may be configured to penetrate tissue wall surface 166 (e.g., when IMD housing 141 is rotated in second rotary direction W2). Secondary helix 146 (e.g., secondary helical body 156) may be configured to substantially tunnel into tissue wall 164 in a second substantially helical path (e.g., around longitudinal axis LD) when IMD housing 141 is rotated in second rotary direction W2. Secondary helical body 156 may be configured such that secondary helix distal end 162 traverses the second substantially helical path as secondary helix 146 substantially tunnels into tissue wall 164. Secondary helical body 156 may be configured such that secondary helix distal end 162 translates in distal direction D relative to tissue wall surface 166 as secondary helix distal end 162 traverses the second substantially helical path. Secondary helical body 156 may be configured such that second electrode 147 traverses at least some portion (e.g., substantially all) of the second substantially helical path and/or translates in distal direction D relative to tissue wall surface 166 as secondary helix 146 substantially tunnels into tissue wall 164. Hence, secondary helical body 156 may be configured to increase a depth of second electrode 147 relative to tissue wall surface 166 as IMD housing 141 is rotated in a second rotational direction (e.g., second rotary direction W2).

Secondary helix 146 (e.g., secondary helical body 156) may be configured to substantially retreat from (e.g., to back out of) tissue wall 164 when IMD housing 141 is rotated in first rotary direction W1. In examples, secondary helical body 156 is configured to substantially retreat from tissue wall 164 over the second substantially helical path. Secondary helical body 146 may be configured such that secondary helix distal end 162 and/or second electrode 147 retreats from tissue wall 164 (e.g., over the substantially helical path) as secondary helix 146 retreats from tissue wall 164. For example, secondary helical body 156 may be configured such that secondary helix distal end 162 and/or second electrode 147 translate in proximal direction D relative to tissue wall surface 166 as secondary helix distal end 162 retreats from tissue wall 164 (e.g., as secondary helix distal end 162 traverses the second substantially helical path). Hence, second helical body 156 may be configured to decrease a depth of second electrode 147 relative to tissue wall surface 166 as IMD housing 141 is rotated in a first rotational direction (e.g., first rotary direction W1).

Thus, IMD 102 may be configured such that a clinician may control a depth of insertion of second electrode 147 relative to tissue wall surface 166 using a direction of rotation and an amount of rotation of IMD housing 141. The clinician may cause IMD housing 141 to rotate in the second rotational direction (e.g., using driver 122 (FIG. 1)) to increase the depth of second electrode 147 relative to tissue wall surface 166. The clinician may cause IMD housing 141 to rotate the first rotational direction (e.g., using driver 122) to decrease the depth of second electrode 147 relative to tissue wall surface 166. The clinician may conduct mapping and/or other sensing using processing circuitry 134 (FIG. 1) as the clinician increases or decreases the depth of second electrode 147 relative to tissue wall surface 166. In examples, IMD 102 is configured to impart a force in distal direction D on secondary helix 146 to assist in causing secondary helix distal end 162 to penetrate tissue wall surface 166 and/or cause secondary helical body 156 to substantially tunnel into tissue wall 164 in the second substantially helical path. For example, IMD housing 141 may be configured to receive a force in distal direction D from driver 122 (e.g., a force imparted by a clinician) and transfer the force in distal direction D to secondary helical body 156.

In examples, IMD 102 includes a support member 168 configured to assist in causing secondary helix 146 (e.g., secondary helix distal end 162) to penetrate tissue wall surface 166 when secondary helix 146 is in contact with tissue wall surface 166 and IMD housing 141 rotates in the second rotational direction (e.g., second rotary direction W2). In examples, support member 168 is configured to cause at least some portion of secondary helical body 156 (e.g., at least secondary helix distal portion 158 and/or secondary helix distal end 162) to remain distally displaced from IMD housing 141 (e.g., IMD distal surface 138) such that, for example, secondary helix distal end 162 tends to penetrate tissue wall 164 and/or tissue wall surface 166 when IMD housing 141 is rotated in the second rotational direction (e.g., second rotary direction W2). Support member 168 may be configured to limit and/or substantially minimize a tendency of secondary helix distal portion 158 and/or secondary helix distal end 162 to establish a position substantially flush against IMD distal surface 138. Such a substantially flush position might otherwise introduce difficulty in causing secondary helix distal end 162 to penetrate tissue wall 164 and/or tissue wall surface 166 using a rotation of IMD housing 141 in the second rotational direction.

Support member 168 may be configured to cause at least secondary helix distal portion 158 and/or secondary helix distal end 162 to remain displaced in distal direction D beyond IMD distal surface 138 when a force in proximal direction P is imparted on secondary helical body 156. For example, support member 168 may be configured to cause secondary helix distal portion 158 and/or secondary helix distal end 162 to remain displaced in distal direction D beyond IMD distal surface 138 when tissue wall surface 166 exerts a proximally-directed force FP (FIG. 6) on secondary helical body 156 (e.g., when secondary helical body 156 contacts tissue wall surface 166). In examples, support member 168 is configured to counteract (e.g., to stop) a displacement of secondary helical body 156 (e.g., secondary helix distal portion 158 and/or secondary helix distal end 162) toward IMD distal surface 138 when the force in proximal direction P (e.g., force FP) is imparted to secondary helical body 156.

In examples, support member 168 is configured to exert a force in the distal direction D on secondary helical body 156 when secondary helical body receives the force in the proximal direction P. For example, support member 168 may be configured to exert a reaction force FR opposite force FP on secondary helical body 156 when secondary helical body receives the force FP. The reaction force FR may substantially counteract the force FP to ceases a displacement of secondary helical body 156 toward IMD distal surface 138 (e.g., a displacement caused by contact with tissue wall surface 166 as, e.g., IMD housing rotates in first rotary direction W1).

Support member 168 may be configured to contact secondary helical body 156 to impart the reaction force FR. In some examples, support member 168 is configured to contact secondary helical body 156 as a result of movement of at least some portion of secondary helical body 156 (e.g., secondary helix distal portion 158 and/or secondary helix distal end 162) toward IMD distal surface 138. For example, IMD 102 may be configured such that gap G (FIG. 4) is present between support member 168 and secondary helix distal end 162 when secondary helix 146 is in the substantially zero-stress position (e.g., in the absence of force FP). Support member 168 may be configured to contact secondary helical body 156 as a result of secondary helical distal end 162 moving toward IMD distal surface 138 substantially over the gap G (e.g., as a result of force FP). In examples, gap G defines a displacement substantially parallel to distal direction D and/or longitudinal axis LD.

In some examples, support member 168 may be configured to substantially maintain contact with secondary helical body 156 even in the absence of movement of some portion of secondary helical body 156 toward IMD distal surface 138. For example, support member 168 may be configured to substantially maintain contact with secondary helical body 156 when secondary helix 146 is in the substantially zero-stress position (e.g., in the absence of force FP). In such cases, support member 168 may be configured to extend from and beyond IMD distal surface 138 to substantially maintain contact with secondary helical body 156 when secondary helix 146 is in the substantially zero-stress position.

In examples, support member 168 defines a bearing surface 170 configured to contact secondary helical body 156. In examples, bearing surface 170 defines a distal-most point of support member 168. In examples, bearing surface 170 is configured to impart reaction for force FR to secondary helical body. Support member 168 may be configured such that bearing surface 170 is displaced from IMD distal surface 138 in distal direction D. For example, support member 168 may be configured to define a displacement D1 (FIG. 4) between bearing surface 170 and IMD distal surface 138. In examples, displacement D1 is substantially parallel to distal direction D and/or longitudinal axis LD.

Support member 168 (e.g., bearing surface 170) may be configured to contact any portion of secondary helical body 156 to impart reaction force FR on secondary helical body 156. For example, in some examples, support member 168 may be configured to contact secondary helix distal portion 158 to impart reaction force FR. In some examples (e.g., when secondary helix 146 completes more than one turn about longitudinal axis LD, and/or when secondary helical body 156 has a sufficient stiffness), support member 168 may be configured to contact secondary helix proximal portion 158 to impart reaction force FR. In some examples, support member 168 may be a substantially separate component affixed to and supported by IMD housing 141 (e.g., IMD distal surface 138). In some examples, support member 168 may be substantially contiguous with IMD housing 141 (e.g., IMD distal surface 138), such that support member 168 and IMD housing 141 define a unified component.

In some examples, support member 168 may be configured to remain substantially stationary with respect to housing distal portion 142 (e.g., IMD distal surface 138). For example, support member 168 may be configured to remain substantially stationary with respect to IMD distal surface 138 as secondary helix distal end 162 moves toward IMD distal surface 138 (e.g., as secondary helical body 156 is compressed toward housing distal portion 142). In examples, support member 168 is configured to substantially maintain displacement D1 between bearing surface 170 and IMD distal surface 138 when secondary helical body 156 moves relative to IMD distal surface 138 (e.g., when secondary helical body 156 is compressed toward housing distal portion 142). Support member 168 may have any configuration sufficient to assist in causing secondary helix 146 (e.g., secondary helix distal end 162) to penetrate tissue wall surface 166 when secondary helix 146 is in contact with tissue wall surface 166 and IMD housing 141 rotates in the second rotational direction (e.g., second rotary direction W2).

In some examples, support member 168 may be configured to move with respect to housing distal portion 142 (e.g., IMD distal surface 138) when support member 168 imparts reaction force FR (FIG. 6) on secondary helical body 156. For example, support member 168 may be configured to deform or bend (e.g., deform or bend toward IMD distal surface 138) when secondary helical body 156 imparts at least some portion of proximally-directed force FP on support member 168. For example, support member 168 may be configured to deform or bend from an initial position with respect to IMD distal portion 138 (“support member initial position) to a stressed position of with respect to IMD distal portion 138 (“support member stressed position”) when secondary helical body 156 imparts the portion of proximally-directed force FP on support member 168.

In examples, support member 168 is resiliently biased, such that support member 168 has a tendency to return or attempt to return to the support member initial position when support member 168 is temporarily displaced (e.g., departs from) from the support member initial position. Support member 168 may be configured such that, when the portion of proximally-directed force FP is exerted on support member 168 and causes support member 168 to deform or bend in proximal direction P toward IMD distal surface 138, a material comprising support member 168 (e.g., a shape memory material) urges support member 168 to return or attempt to return to the support member initial position. In examples, the support member initial position is a substantially zero-stress position, where any stresses on support member 168 arise from properties or phenomena internal to support member 168, such as mass, internal temperature, residual stresses (e.g., from fabrication and/or mechanical attachment to another component), and the like.

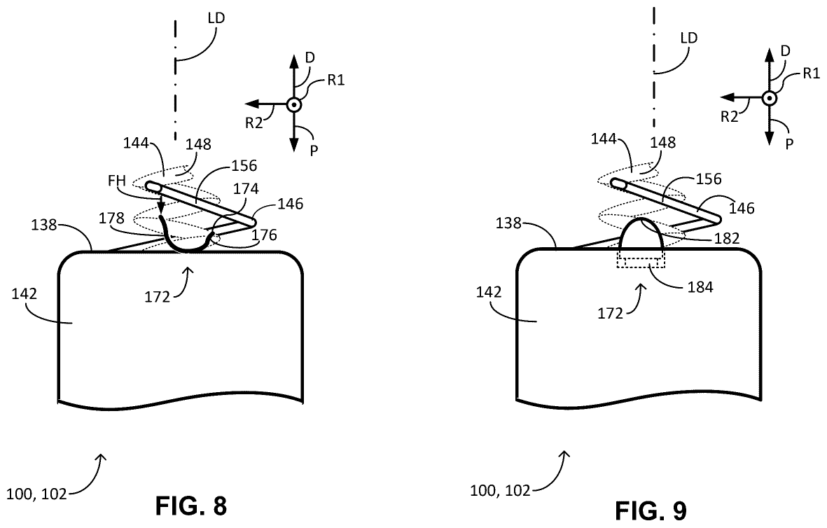

FIG. 8 is a schematic view of a portion of IMD 102 including a support member 172 including a bearing surface 174. Support member 172 is an example of support member 168. Bearing surface 174 is an example of bearing surface 170.

Support member 172 may include a first portion 176 configured to contact secondary helical body 156 configured to substantially maintain secondary helix distal portion 158 displaced from IMD distal surface 138 (e.g., displaced in distal direction D). In examples, first portion 176 (e.g., bearing surface 174) is configured to contact secondary helical body when a force FH in proximal direction P is imparted on support member 172. In examples, secondary helical body 156 is configured to impart force FH on support member 172 (e.g., when secondary helical body 156 is compressed toward IMD distal surface 138). At least first portion 176 may be configured to move relative to housing distal portion 142 (e.g., IMD distal surface 138) when force FH is imparted on support member 172.

In some examples, support member 172 includes a second portion 178 configured to cause first portion 176 to move relative to housing distal portion 142 when force FH is imparted on second portion 178. For example, support member 172 may substantially be a rocker arm configured to pivot relative to IMD distal surface 138 when force FH acts on support member 172. Support member 172 may be configured such that first portion 176 moves toward secondary helical body 156 when second portion 178 moves toward IMD distal surface 138 (e.g., when force FH causes second portion 178 to move toward IMD distal surface). Support member 172 may be configured to substantially pivot relative to IMD distal surface 138 when first portion 176 moves toward secondary helical body 156 and second portion 178 moves toward IMD distal surface 138.

FIG. 9 is a schematic view of a portion of IMD 102 including a support member 180 including a bearing surface 182. Support member 180 is an example of support member 168, 172. Bearing surface 182 is an example of bearing surface 170, 174. Support member 180 may include a compressible member 184 (depicted in dashed lines) configured to deform when support member 180 moves in proximal direction P relative to housing distal portion 142 (e.g., IMD distal surface 138). In examples, compressible member 184 is a substantially elastically deforming element (e.g., a spring or other substantially elastically deforming element) which exhibits a change in shape when a compressive force is applied to compressible member 184 (e.g., a force in proximal direction P), and which substantially reverses the change in shape when the compressive force is removed. Support member 180 may be configured such that bearing surface 182 moves in proximal direction P (e.g., toward IMD distal surface 138) when the compressive force is applied to compressible member 184. Support member 180 may be configured such that bearing surface 182 moves in distal direction D (e.g., away from IMD distal surface 138) when the compressive force is removed from compressible member 184. In examples, IMD housing 141 (e.g., housing distal portion 142 and/or IMD distal surface 138) supports compressible member 184. Support member 180 may be configured such that, when the force in proximal direction P is imparted to compressible member 184, housing distal portion 142 and/or IMD distal surface 138 impart a reaction force on compressible member 184 in distal direction D.

In some examples, secondary helix 146 (e.g., secondary helical body 156) is configured to bend and/or alter its orientation with respect to IMD housing 141 and/or IMD distal surface 138 when a torque in the second rotational direction (e.g., second rotary direction W2) is imparted to IMD housing 141. For example, secondary helix 146 may be configured to bend and/or alter its orientation when secondary helix 146 reaches a threshold travel within tissue wall 164 and the torque is imparted to IMD housing 141 in the second rotational direction. The bending and/or altering of its orientation may allow removal of both primary helix 144 and secondary helix 146 from tissue wall 164 using rotation of IMD housing 141 in the second rotational direction.

For example, as discussed, secondary helix 146 may be configured to embed within (e.g., travel into) tissue wall 164 when IMD housing is rotated in second rotary direction W2, and configured to withdraw from (e.g., travel out of) tissue wall 164 when IMD housing is rotated in first rotary direction W1. In contrast, primary helix 144 may be configured to embed within (e.g., travel into) tissue wall 164 when IMD housing is rotated in first rotary direction W1, and configured to withdraw from (e.g., travel out of) tissue wall 164 when IMD housing is rotated in second rotary direction W2. Secondary helix 146 may be configured such that, with both primary helix 144 and secondary helix 146 embedded within tissue wall 164, imparting a torque of IMD housing 141 in second rotary direction W2 (e.g., to withdraw primary helix 144) causes IMD housing 141 to impart a bending force on secondary helix 146 sufficient to cause secondary helix 146 to depart from its helical shape. For example, IMD housing 141 may impart the bending force when secondary helix 146 has substantially reached its full travel into tissue wall 164. Secondary helix 146 may be configured such that, as IMD housing 141 continues to rotate in second rotary direction W2 (e.g., with secondary helix 146 at its full travel), primary helix 144 continues to withdraw from tissue wall 164 as secondary helix proximal portion 160 bends to substantially pull the embedded portion of secondary helix 146 out of tissue wall 164.

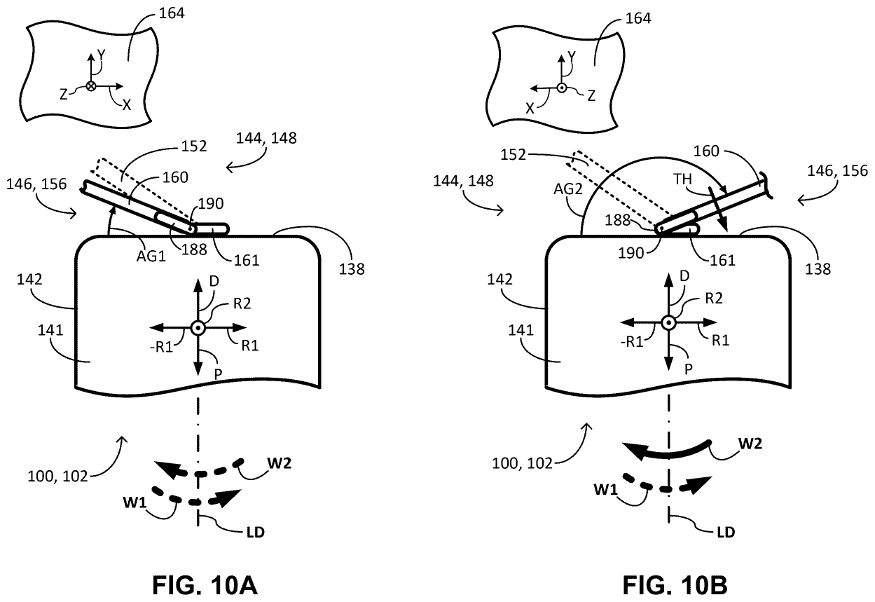

As an example, FIG. 10A depicts a portion of IMD 102 with secondary helix proximal portion 160 extending from secondary helix base portion 161 and embedded within tissue wall 164. Primary helix proximal portion 152 (depicted in dashed lines) is also embedded within tissue wall 164. In FIG. 10A, tissue wall 164 defines the X-Y-Z coordinate system with the Z-axis proceeding into the page. FIG. 10B depicts the portion of IMD 102 having rotated in the rotary direction W2 with respect to tissue wall 164. In FIG. 10B, IMD 102 has rotated relative to tissue wall 164 such that the Z-axis proceeds out of the page.

FIG. 10A depicts IMD 102 in a static orientation with IMD 102 substantially stationary with respect to tissue wall 164. Some portion of primary helix 144 and primary helix proximal portion 152 have been caused to embed within tissue wall 164 using rotation of IMD housing 141 in first rotary direction W1. Some portion of secondary helix 146 and secondary helix proximal portion 160 have been caused to embed within tissue wall 164 using rotation of IMD housing 141 in second rotary direction W2 (e.g., a rotation in second rotary direction W2 subsequent to the prior rotation in first rotary direction W1). In FIG. 10A, secondary helix proximal portion 160 defines a first orientation with respect to the R1-D-R2 coordinate system of IMD 102.

In the first orientation, secondary helix proximal portion 160 extends from secondary helix base portion 161 in a direction opposite the R1 direction of the R1-D-R2 coordinate system (e.g., the −R1 direction). Stated similarly, in the first orientation, secondary helix proximal portion 160 extends from secondary helix base portion 161 in a direction causing secondary helix proximal portion 160 to define a projection (e.g., a dot product) onto a −R1 axis defining the −R1 direction. In examples, in the first orientation, secondary helix proximal portion 160 defines a first angle AG1 between secondary helix proximal portion 160 and IMD distal surface 138. In examples, first angle AG1 defines a first angular displacement between secondary helix proximal portion 160 and IMD distal surface 138 measures in a particular rotational direction (e.g., a clockwise direction about the R2 axis).

As discussed, when primary helix proximal portion 152 and secondary helix proximal portion 160 substantially embedded within tissue wall 164, IMD housing 141 might be rotated in second rotary direction W2 in order to, for example, cause primary helix 144 to withdraw from tissue wall 164 (e.g., to travel out of tissue wall 164 in proximal direction P). Secondary helical body 156 may be configured to bend and/or alter its orientation with respect to IMD housing 141 and/or IMD distal surface 138 when IMD housing 141 is caused (e.g., by a torque in second rotary direction W2) to rotate in second rotary direction W2.

For example, FIG. 10B depicts IMD housing 141 having rotated in second rotary direction W2 about 180 degrees with respect to tissue wall 164, as compared to FIG. 10A. In FIG. 10B, secondary helix proximal portion 160 has altered its orientation to define a second orientation with respect to the R1-D-R2 coordinate system, such that secondary helix proximal portion 160 extends from secondary helix base portion 161 in the R1 direction of the R1-D-R2 coordinate system. Stated similarly, in the second orientation, secondary helix proximal portion 160 extends from secondary helix base portion 161 in a direction causing secondary helix proximal portion 160 to define a projection (e.g., a dot product) onto the R1 axis of the R1-D-R2. In examples, in the second orientation, secondary helix proximal portion 160 defines an second angle AG2 between secondary helix proximal portion 160 and IMD distal surface 138. In examples, second angle AG2 defines a second angular displacement between secondary helix proximal portion 160 and IMD distal surface 138 measured in the particular rotational direction of first angle AG1.

In examples, secondary helical body 156 is configured to transition from the first orientation of FIG. 10A to the second orientation of FIG. 10B by a torque imparted on IMD housing 141 (e.g., a torque about longitudinal axis LD in second rotary direction W2). IMD housing 141 may be configured to transfer at least some portion of the torque to secondary helix base portion 161, such that both IMD housing 141 and secondary helix base portion 161 rotate about longitudinal axis LD. IMD housing 141 may be configured to cause a torque TH (FIG. 10B) around secondary helix base portion 161 on secondary helix proximal portion 160 when IMD housing 141 transfers the torque about longitudinal axis LD to secondary helix base portion 161. For example, when secondary helix proximal portion 160 is at least partially embedded in tissue wall 164 and secondary helix base portion 161 is caused to rotate relative to tissue wall 164 (e.g., by a rotation of IMD housing 141), contact between secondary helix proximal portion 160 and tissue wall 164 may cause secondary helix proximal portion 160 to experience the torque TH. In some examples, tissue wall 164 may substantially impart a force on secondary helix proximal portion 160 when secondary helix base portion 161 rotates relative to tissue wall 164, such that secondary helix proximal portion 160 experiences the torque TH.

In examples, when secondary helical body 156 bends and/or alters its orientation with respect to IMD housing 141 and/or IMD distal surface 138 in response to a rotation in the second rotational direction (e.g., second rotary direction W2), at least secondary helix proximal portion 160 transitions from a first orientation defining a first projection onto a first axis in a first direction (e.g., the −R1 axis in the −R1 direction) to a second orientation defining a second projection onto a second axis in a second direction opposite the first direction (e.g., the R1 axis in the R1 direction). In examples, when secondary helical body 156 bends and/or alters its orientation with respect to IMD housing 141 and/or IMD distal surface 138 in response to a rotation in the second rotational direction (e.g., second rotary direction W2), at least secondary helix proximal portion 160 increases an angular displacement (e.g., from the first angular displacement of AG1 to the second angular displacement of AG2) between secondary helix proximal portion 160 and IMD distal surface 138.

In some examples, secondary helix proximal portion 160 is configured to bend when secondary helix proximal portion 160 transitions from the first configuration to the second configuration. For example, secondary helix proximal portion 160 may be configured to bend (e.g., define and/or alter a curvature in the D-R1 plane) in the vicinity of secondary helix base portion 161 when secondary helix proximal portion 160 transitions from the first configuration to the second configuration. In some examples, secondary helical body 156 has a bending stiffness less than a bending stiffness of first helical body 148. In examples, secondary helical body 156 has a greater flexibility than first helical body 148, such that, for example, a particular value of torque in second rotary direction W2 causes secondary helix proximal portion 160 to bend (e.g., bend in the vicinity of secondary helix base portion 161), but the particular value of torque when applied in first rotary direction W1 is insufficient to cause bending of primary helix proximal portion 152 (e.g., bending in the vicinity of primary helix base portion 153 (FIG. 3)).

In some examples, secondary helix 146 (e.g., secondary helix base portion 161) may include a mechanism 188 configured to allow secondary helix proximal portion 160 to transition from the first configuration to the second configuration. Mechanism 188 may be configured to limit and/or mitigate bending of secondary helix proximal portion 160 when secondary helix proximal portion 160 transitions from the first configuration to the second configuration. In some examples, mechanism 188 includes a hinge 190 configured to allow secondary helix proximal portion 160 to transition from the first configuration to the second configuration. For example, mechanism 188 and/or hinge 190 may be configured to permit a rotation of secondary helix proximal portion 160 about a radial axis defined by IMD housing 141 (e.g., axis R2) when secondary helix proximal portion 160 transitions from the first configuration to the second configuration.

Secondary helix proximal portion 160 may be configured to transition from imparting a pushing force on secondary helix distal portion 158 to imparting a pulling force on secondary helix distal portion 158 when secondary helix proximal portion 160 transitions from the first orientation to the second orientation. The pulling force may substantially cause secondary helix distal portion 158 and/or secondary helix distal end 162 to withdraw from tissue wall 164 (e.g., to displace within tissue wall 164 in proximal direction P). Secondary helical body 156 may be configured to cause secondary helix distal portion 158 and/or secondary helix distal end 162 to withdraw from tissue wall 164 in a substantially atraumatic manner when secondary helix proximal portion 160 imparts the pulling force. In examples, secondary helical body 156 has a sufficient flexibility such that secondary helix distal portion 158 and/or secondary helix distal end 162 substantially follows first helical path P1 (FIG. 3) when secondary helix proximal portion 160 imparts the pulling force on secondary helix distal portion 158 and/or secondary helix distal end 162.

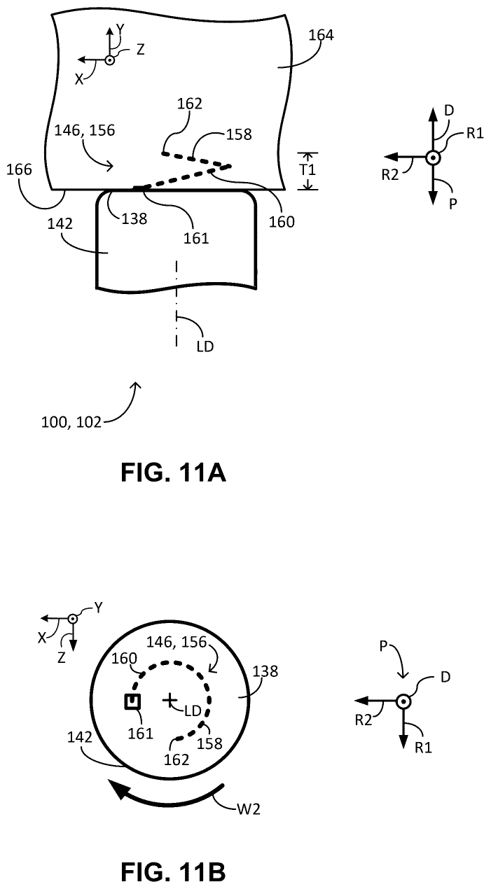

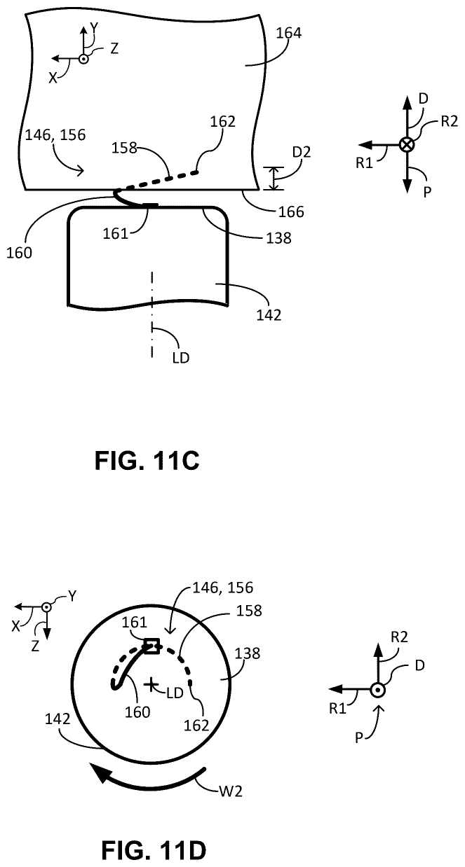

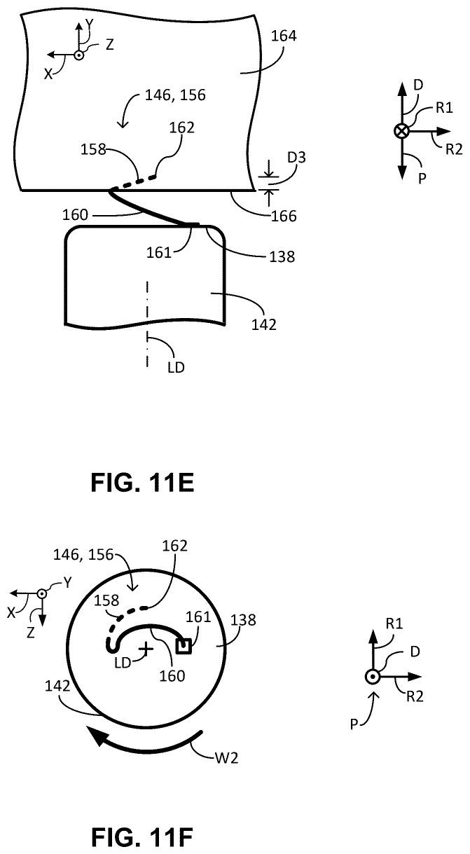

For example, FIG. 11A depicts a portion of IMD 102 with secondary helix 146 embedded within tissue wall 164 to a travel length T1. Travel length T1 may be the full travel length of secondary helix 146 when IMD housing 141 is rotated in the second rotational direction (e.g., second rotary direction W2). In examples, travel length T1 is substantially equal to second helical height H2 (FIG. 4) and/or substantially parallel to longitudinal axis LD. In some examples, travel length T1 is substantially equal to a displacement extending from secondary helix distal end 162 to secondary helix base portion 161 and substantially parallel to longitudinal axis LD. In examples, IMD 102 is configured such that at least some portion of IMD housing 141 (e.g., IMD distal surface 138) contacts tissue wall surface 166 when secondary helix 146 is embedded within tissue wall 164 to travel length T1. FIG. 11B depicts an end view of the portion of IMD 102 of FIG. 11A. The portion of secondary helix 146 embedded within tissue wall 164 is depicted with dashed lines in FIG. 11A and FIG. 11B. In FIG. 11A, secondary helix proximal portion 160 is in the first orientation (FIG. 10A).

FIG. 11C depicts IMD housing 141 having rotated in second rotary direction W2 about 90 degrees with respect to tissue wall 164, as compared to FIG. 11A and FIG. 11B. In FIG. 11C, secondary helix proximal portion 160 has altered its orientation from the first orientation to the second orientation (FIG. 10B). Secondary helix proximal portion 160 has transitioned to imparting a pulling force on secondary helix distal portion 158 as IMD housing 141 rotates in second rotary direction W2. The pulling force has caused secondary helix distal portion 158 and/or secondary helix distal end 162 to withdraw from tissue wall 164 (e.g., substantially over first helical path P1 (FIG. 3), such that secondary helical body 156 defines a distance D2 between secondary helix distal end 162 and tissue wall surface 166 which is less than travel length T1. FIG. 11D depicts an end view of the portion of IMD 102 of FIG. 11C, with the portion of secondary helix 146 embedded within tissue wall 164 depicted with dashed lines in FIG. 11C and FIG. 11D.

FIG. 11E depicts IMD housing 141 having rotated in second rotary direction W2 a further about 90 degrees with respect to tissue wall 164, as compared to FIG. 11C and FIG. 11D. In FIG. 11E, secondary helix proximal portion 160 continues to impart the pulling force on secondary helix distal portion 158 as IMD housing 141 rotates in second rotary direction W2. The pulling force has caused secondary helix distal portion 158 and/or secondary helix distal end 162 to withdraw from tissue wall 164 (e.g., substantially over first helical path P1 (FIG. 3), such that secondary helical body 156 defines a distance D3 between secondary helix distal end 162 and tissue wall surface 166. Distance D3 is less than distance D2. FIG. 11F depicts an end view of the portion of IMD 102 of FIG. 11E, with the portion of secondary helix 146 embedded within tissue wall 164 depicted with dashed lines in FIG. 11E and FIG. 11F.

Hence, IMD 102 may be configured such that primary helical body 148 tends to embed within tissue wall 164 when IMD housing 141 is rotated in the first rotational direction (e.g., first rotary direction W1) and tends to withdraw from tissue wall 164 when IMD housing 141 is rotated in the second rotational direction opposite the first rotational direction (e.g., second rotary direction W2). IMD 102 may be configured such that secondary helical body 156 tends to embed within tissue wall 164 when IMD housing 141 is rotated in the second rotational direction and tends to withdraw from tissue wall 164 when IMD housing 141 is rotated in the first rotational direction. Secondary helical body 156 may be configured to bend and/or alter its orientation with respect to IMD housing 141 and/or IMD distal surface 138 when secondary helix 146 reaches a threshold travel within tissue wall 164 (e.g., a travel of about travel length T1) and the torque is imparted to IMD housing 141 in the second rotational direction to, for example, cause primary helical body 148 to withdraw from tissue wall 164. Secondary helix proximal portion 160 may be configured to transition from imparting a pushing force on secondary helix distal portion 158 to imparting a pulling force on secondary helix distal portion 158 cause secondary helix distal portion 158 to withdraw from tissue wall 164 as the torque is imparted to IMD housing 141 in the second rotational direction.

Medical system 100 (e.g., IMD 102) may comprise a pacemaker such as a leadless and/or wholly intracardiac pacemaker. One or more of electrodes 139, 145, 147 may be electrically connected to processing circuitry 134. Processing circuitry 134 may be operably connected to operating circuitry configured to deliver therapy to a patient and/or sense physiological signals of the patient using electrodes 139, 145, 147. In examples, IMD 102 includes a retrieval structure 192 configured to releasably couple with delivery system 120 (e.g., head portion 128) to assist in, for example, delivery, deployment, and/or retrieval of IMD 102. In some examples, retrieval structure 192 defines a proximal end 194 of IMD housing 141.

Processing circuitry 134 may include fixed function circuitry and/or programmable operating circuitry. In examples, processing circuitry 134 includes circuitry configured to perform one or more functions of operating circuitry, such as therapy delivery circuitry, sensing circuitry, processing circuitry, switching circuitry, communication circuitry, and/or other circuitries. Processing circuitry 134, as well as other processors, operating circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 134 includes multiple components, such as any combination of one or more microprocessors, one or more DSPs, one or more ASICs, or one or more FPGAs, as well as other discrete or integrated logic circuitry, and/or analog circuitry.

Functions attributed to processing circuitry 134 may be embodied as software, firmware, hardware or any combination thereof. Processing circuitry 134 may include, for instance, a variety of capacitors, transformers, switches, and the like configured to perform the functions of processing circuitry 134. In examples, processing circuitry 134 may be configured to communicate with another device, such as a patient input/output device, a clinician input/output device, and/or others. Processing circuitry 134 may include any suitable hardware, firmware, software or any combination thereof for communicating with another device. In addition, processing circuitry 134 may communicate with a networked computing device and a computer network. In examples, processing circuitry 134 and/or other circuitry of medical system 100 is configured to deliver stimulation signals to and/or receive sensing signals from electrodes 139, 145, 147, and/or other electrodes and/or sensors within medical system 100 or external to medical system 100. Processing circuitry 134 may be configured to provide electrical signals, e.g., pacing therapy, to electrodes 139, 145, 147, and/or other electrodes within medical system 100. Processing circuitry 134 may be configured to receive electrical signals, e.g., sensed cardiac electrical signals, from electrodes 139, 145, 147, and/or other electrodes within medical system 100.

Medical system 100 (e.g., processing circuitry 134) can also include a memory configured to store program instructions, such as software, which may include one or more program modules, which are executable by processing circuitry 134. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by processing circuitry 134 cause processing circuitry 134 to perform various functions described herein and/or other functions of processing circuitry 134.

IMD housing 141 may enclose processing circuitry 134 and/or other circuitry within medical system 100. IMD housing 141 may be configured to fluidly isolate processing circuitry 134 and/or other circuitry from an environment in contact with an exterior surface of IMD housing 141. In examples, IMD housing 141 is configured to hermetically seal an enclosure defined by IMD 102 and holding processing circuitry 134 and/or other circuitry. IMD housing 141 may be configured to define shapes that are easily accepted by the patient's body while minimizing patient discomfort. For example, portions of IMD housing 141 (e.g., housing distal portion 142 and/or a proximal portion of IMD housing 141) may define a substantially cylindrical shape with cylindrical sidewalls. In other examples, portions of IMD housing 141 may define substantially rectangular or other non-cylindrical shapes. IMD housing 141 may define shapes in which corners and edges are designed with relatively large radii, in order to present a housing having smoothly contoured exterior surfaces. In examples, attachment member 132 (e.g., primary helical body 148 and/or secondary helical body 156) is coupled to IMD housing 141.

Communication links 135, 137 may be hard-line and/or wireless communications links. In some examples, communication links 135, 137 may comprise some portion of processing circuitry 134. In some examples, communication links 135, 137 comprise a wired connection, a wireless Internet connection, a direct wireless connection such as wireless LAN, Bluetooth™, Wi-Fi™, and/or an infrared connection. Communication links 135, 137 may utilize any wireless or remote communication protocol.

As used here, when a first portion of a system (e.g., medical system 100) is substantially parallel to a second portion of or an axis defined by the system, this may mean the first portion is parallel or nearly parallel to the second portion or the axis to the extent permitted by manufacturing tolerances. In some examples, when the first portion is substantially parallel to the second portion or the axis, this may mean a first vector defined by the first component of the system defines an angle of less than 10 degrees, in some examples less than 5 degrees, and in some examples less than 1 degree, with a second vector defined by the second component or the axis. When a first portion of the system is substantially perpendicular to a second portion of or an axis defined by the system, this may mean the first portion is perpendicular or nearly perpendicular to the second portion or the axis to the extent permitted by manufacturing tolerances. In some examples, when the first portion is substantially perpendicular to the second portion or the axis, this may mean that the first vector defined by the first component of the system defines an angle of at least 80 degrees, in some examples at least 85 degrees, and in some examples at least 89 degrees, with the second vector defined by the second component.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

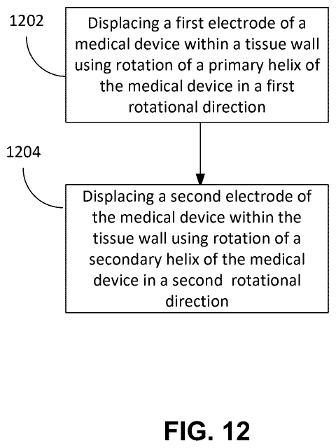

A technique for holding an implantable medical device within a receptacle device is illustrated in FIG. 12. Although the technique is described mainly with reference to medical system 100 of FIGS. 1-11, the technique may be applied to other medical systems in other examples.

The technique includes displacing, using an IMD housing 141 of an IMD 102, a first electrode 145 within a tissue wall 164 of a patient using a rotation of a primary helix 144 of IMD 102 in a first rotational direction (1202). In examples, the technique includes causing the rotation of primary helix 144 by imparting a torque in the first rotational direction to IMD housing 141. A delivery system 120 may impart the torque in the first rotational direction to IMD housing 141. In examples, a driver 122 of delivery system 120 imparts the torque in the first rotational direction to a head portion 128 of delivery system 120. Head portion 128 may transfer the torque in the first rotational direction to IMD housing 141.

The torque imparted to IMD housing 141 in the first rotational direction may cause a rotation of IMD housing 141 in the first rotational direction. The rotation of IMD housing 141 in the first rotational direction may cause the rotation of primary helix 144 in the first rotational direction. In examples, primary helix 144 rotates about a longitudinal axis LD defined by IMD 102 when IMD housing 141 rotates about longitudinal axis LD.

Primary helix 144 may support first electrode 145. In examples, the rotation of primary helix 144 in the first rotational direction causes first electrode 145 to embed within tissue wall 164. In examples, the rotation of primary helix 144 causes a primary helix distal end 154 to displace in a distal direction D from a tissue wall surface 166 of tissue wall 164. In some examples, the rotation of primary helix 144 causes first electrode 145 to displace in the distal direction D from tissue wall surface 166. In some examples, primary helix distal end 154 and/or first electrode 145 embed within tissue wall surface 164 over a first helical path P1 defined by primary helix 144.

The technique includes displacing, using IMD housing 141, a second electrode 147 within tissue wall 164 using a rotation of a secondary helix 146 of IMD 102 in a second rotational direction opposite the first rotational direction (1204). In examples, the technique includes causing the rotation of secondary helix 146 by imparting a torque in the second rotational direction to IMD housing 141. Delivery system 120 may impart the torque in the second rotational direction to IMD housing 141. In examples, driver 122 imparts the torque in the second rotational direction to head portion 128. Head portion 128 may transfer the torque in the second rotational direction to IMD housing 141.

The torque imparted to IMD housing 141 in the second rotational direction may cause a rotation of IMD housing 141 in the second rotational direction. The rotation of IMD housing 141 in the second rotational direction may cause the rotation of secondary helix 146 in the second rotational direction. In examples, secondary helix 146 rotates about longitudinal axis LD when IMD housing 141 and/or primary helix 144 rotates about longitudinal axis LD.

Secondary helix 146 may support second electrode 147. In examples, the rotation of secondary helix 146 in the second rotational direction causes second electrode 147 to embed within tissue wall 164. In examples, the rotation of secondary helix 146 causes a secondary helix distal end 162 to displace in distal direction D from tissue wall surface 166. In some examples, the rotation of secondary helix 146 causes second electrode 147 to displace in distal direction D from tissue wall surface 166. In some examples, secondary helix distal end 162 and/or second electrode 145 embed within tissue wall surface 164 over a second helical path P2 defined by primary helix 144. In examples, primary helix 144 defines one of a right-handed helix or a left-handed helix and secondary helix 146 defines the other the right-handed helix or the left-handed helix. The technique may include providing and/or sensing, using processing circuitry 134, a signal using at least one of first electrode 145 or second electrode 147.

The technique may include embedding secondary helix 146 within tissue wall 164 when primary helix 144 is embedded in tissue wall 164. In examples, a support member 168 assists secondary helix 146 (e.g., secondary helix distal end 162) in penetrating tissue wall surface 166 when primary helix 144 is embedded with tissue wall 164. Support member 168 may cause at least some portion of a secondary helical body 156 of secondary helix 146 (e.g., at least secondary helix distal portion 158 and/or secondary helix distal end 162) to remain distally displaced from an IMD distal surface 138 of IMD housing 141. Support member 168 may limit and/or substantially minimize a tendency of secondary helix distal portion 158 and/or secondary helix distal end 162 to establish a position substantially flush against IMD distal surface 138.

In examples, support member 168 causes secondary helix distal portion 158 and/or secondary helix distal end 162 to remain displaced in distal direction D beyond IMD distal surface 138 when tissue wall surface 166 exerts a proximally-directed force FP on secondary helical body 156. In examples, support member 168 counteracts (e.g., stops) a displacement of secondary helical body 156 toward IMD distal surface 138 when force FP is imparted to secondary helical body 156. Support member 168 may exert a reaction force FR in the distal direction D on secondary helical body 156 when secondary helical body 156 receives the force FP. In examples, support member 168 contacts secondary helical body 156 to impart the reaction force FR.

In some examples, secondary helix 146 bends and/or alters its orientation with respect to IMD housing 141 and/or IMD distal surface 138 when the torque in the second rotational direction is imparted to IMD housing 141. Secondary helix 146 may bend and/or alter its orientation when secondary helix 146 reaches a threshold travel within tissue wall 164 and the torque is imparted to IMD housing 141 in the second rotational direction. In examples, secondary helix distal end 162 moves in proximal direction P toward tissue wall surface 166 when secondary helix 146 bends and/or alters its orientation and IMD housing 141 rotates in the second rotational direction. In examples, a secondary helix proximal portion 160 transitions from a pushing force on a secondary helix distal portion 158 to a pulling force on secondary helix distal portion when secondary helix 146 bends and/or alters its orientation and IMD housing 141 rotates in the second rotational direction. In examples, primary helix distal end 154 moves in proximal direction P toward tissue wall surface 166 when IMD housing 141 rotates in the second rotational direction. In examples, primary helix distal end 154 moves in proximal direction P toward tissue wall surface 166 when secondary helix proximal portion 160 exerts the pulling force on secondary helix distal portion 158.

Various examples of the disclosure have been described. Any combination of the described systems, operations, or functions is contemplated. These and other examples are within the scope of the following claims.

The techniques of this disclosure may also be described in the following examples.

Example 1: A medical device configured to be positioned within an anatomical volume defined by a body of a patient, the medical device comprising:
a primary helix surrounding a longitudinal axis defined by a housing of the medical device, wherein the primary helix extends in a distal direction beyond a distal portion of the housing, wherein the primary helix supports a first electrode, and wherein the primary helix is configured to displace the first electrode within a tissue wall of a patient when a torque in a first rotational direction about the longitudinal axis is imparted to a housing;
a secondary helix surrounding the longitudinal axis, wherein the secondary helix extends in the distal direction beyond the distal portion of the housing, wherein the secondary helix supports a second electrode, and wherein the secondary helix is configured to displace the second electrode within the tissue wall when a torque in a second rotational direction about the longitudinal axis is imparted to the housing; and
processing circuitry electrically connected to the first electrode and the second electrode, wherein the processing circuitry is configured to at least one of provide a signal or sense a signal using at least one of the first electrode or the second electrode.

Example 2: The medical device of Example 1, wherein a distal portion of the primary helix supports the first electrode.

Example 3: The medical device of Example 2, wherein the primary helix defines a first helical path from the distal portion of the housing to a distal end of the primary helix and defines a midpoint of the first helical path substantially halfway between the distal portion of the housing and the distal end of the primary helix, and wherein the distal portion of the primary helix extends from the midpoint of the first helical path to the distal end of the primary helix.

Example 4: The medical device of any of Examples 1-3, wherein a distal portion of the secondary helix supports the second electrode.

Example 5: The medical device of any of Example 4, wherein the secondary helix defines a second helical path from the distal portion of the housing to a distal end of the secondary helix and defines a midpoint of the second helical path substantially halfway between the distal portion of the housing and the distal end of the secondary helix, and wherein the distal portion of the secondary helix extends from the midpoint of the second helical path to the distal end of the secondary helix.

Example 6: The medical device of any of Examples 1-5, wherein:
the distal portion of the housing defines a distal end of the housing,
the primary helix extends defines a first distance from the distal end of the housing to
the distal end of the primary helix,
the secondary helix extends defines a second distance from the distal end of the housing to the distal end of the secondary helix,
the first distance and the second distance are substantially parallel to the longitudinal axis, and the first distance is greater than the second distance.

Example 7: The medical device of any of Examples 1-6, wherein the secondary helix is configured to slidably translate over a surface of the tissue wall when the torque in the first rotational direction is imparted to the housing and the primary helix displaces the first electrode within the tissue wall.

Example 8: The medical device of any of Examples 1-6, wherein the primary helix defines one of a right-handed helix or a left-handed helix and the secondary helix defines the other of the right-handed helix or the left-handed helix.

Example 9: The medical device of Example 8, wherein, when the secondary helix defines the left-handed helix, the secondary helix is configured to slidably translate over a surface of the tissue wall when the housing is rotated in a right-handed direction about the longitudinal axis, and wherein when the secondary helix defines the right-handed helix, the secondary helix is configured to slidably translate over the surface of the tissue wall when the housing is rotated in a left-handed direction about the longitudinal axis.

Example 10: The medical device of any of Examples 1-9, wherein the secondary helix is configured to compress toward the housing when a force in a proximal direction opposite the distal direction is imparted on the secondary helix.

Example 11: The medical device of any of Examples 1-10, wherein the primary helix defines a first number of turns around the longitudinal axis between the distal portion of the housing and the distal end of the primary helix and wherein the secondary helix defines a second number of turns around the longitudinal axis between the distal portion of the housing and the distal end of the secondary helix, wherein the second number of turns is less than or equal to 50% of the second number of turns.

Example 12: The medical device of Example 11, wherein the second number of turns is less than or equal to one turn.

Example 13: The medical device of any of Examples 1-12, further comprising a support member supported by the housing, wherein the support member is configured to cause at least the distal end of the secondary helix to displace in the distal direction beyond the distal end of the housing when a force in the proximal direction is imparted on the secondary helix.

Example 14: The medical device of Example 13, wherein the support member extends in the distal direction beyond the distal end of the housing.

Example 15: The medical device of Example 13 or Example 14, wherein the support member comprises a rocker body including a first portion configured to contact the secondary helix when a force in the proximal direction is imparted on the secondary helix, and including a second portion configured to displace the secondary helix in the distal direction beyond the distal end of the housing when the secondary helix contacts the first portion.

Example 16: The medical device of Example 15, wherein the rocker body is configured to pivot relative to the housing around a pivot point between a first portion of the rocker body and a second portion of the rocker body when the secondary helix imparts at least some portion of the force in the proximal direction on the first portion.

Example 17: The medical device of Example 15 or Example 16, wherein the second portion is configured to impart a force in the distal direction on the secondary helix when the secondary helix imparts at least some portion of the force in the proximal direction on the first portion.

Example 18: The medical device of any of Examples 13-17, wherein the support member includes a distal portion configured to contact the secondary helix when the force in the proximal direction is imparted on the secondary helix and a proximal portion supported by the housing, wherein the distal portion is configured to displace toward the housing at least when the torque in the second rotational direction causes the tissue wall to impart a proximally directed force on the distal portion.

Example 19: The medical device of any of Examples 13-18, wherein the support member includes a compressible device configured to deform when the support member moves in the proximal direction relative to the housing, and wherein the compressible device is configured to impart a force in the distal direction on the secondary helix when the compressible device deforms.

Example 20: The medical device of any of Examples 1-19, wherein the secondary helix includes a proximal portion supported by the housing, and wherein the secondary helix is configured to bend when the torque in the second rotational direction is imparted to the housing and the proximal portion imparts a pulling force on the distal portion.

Example 21: The medical device of any of Examples 1-20, wherein the proximal portion is configured to pivot relative to the housing when the torque in the second rotational direction is imparted to the housing and the secondary helix exceeds a threshold travel within the tissue wall.

Example 22: The medical device of any of Examples 1-21, wherein the secondary helix includes a hinge mechanism configured to allow the proximal portion to pivot relative to the housing.

Example 23: The medical device of any of Examples 1-22, wherein the primary helix defines a first substantially circular helix with respect to the longitudinal axis and the secondary helix defines a second substantially circular helix with respect to the longitudinal axis.

Example 24: The medical device of any of Examples 1-23, wherein the primary helix defines first pitch with respect to the longitudinal axis and the secondary helix defines a second pitch with respect to the longitudinal axis, and wherein the second pitch is greater than or equal to 75% of the first pitch and less than or equal to 125% of the first pitch.

Example 25: The medical device of any of Examples 1-23, wherein the primary helix defines first pitch with respect to the longitudinal axis and the secondary helix defines a second pitch with respect to the longitudinal axis, and wherein the second pitch is less than 80% of the first pitch.

Example 26: The medical device of any of Examples 1-25, wherein the primary helix is between the longitudinal axis and the secondary helix.

Example 27: The medical device of any of Examples 1-26, wherein the secondary helix is between the longitudinal axis and the primary helix.

Example 28: The medical device of any of Examples 1-27, wherein a proximal portion of the housing includes a retrieval structure configured to transfer the torque in the first rotational direction and transfer the torque in the second rotational direction from a driver to the housing.

Example 29: The medical system of any of Examples 1-28, wherein the anatomical volume is defined by a volume of tissue comprising a heart of the patient.

Example 30: The medical system of any of Examples 1-29, wherein the processing circuitry is configured to at least one of provide the signal the heart of the patient or sense the signal from the heart of the patient using the at least one of the first electrode or the second electrode.

Example 31: The medical system of any of Examples 1-30, wherein the housing is configured to be transported through vasculature of the patient.

Example 32: A medical device configured to be positioned within an anatomical volume defined by a body of a patient, the medical device comprising:
a primary helix surrounding a longitudinal axis defined by a housing of the medical device, wherein the primary helix extends in a distal direction beyond a distal portion of the housing, and wherein the primary helix is configured to displace a distal end of primary helix within a tissue wall of a patient when a torque in a first rotational direction about the longitudinal axis is imparted to a housing;
a secondary helix surrounding the longitudinal axis, wherein the secondary helix extends in the distal direction beyond the distal portion of the housing, and wherein the secondary helix is configured to displace a distal end of the secondary helix within the tissue wall when a torque in a second rotational direction about the longitudinal axis and opposite the first rotational direction is imparted to the housing,
wherein at least one of the primary helix or the secondary helix supports an electrode;
a support member supported by the housing, wherein the support member is configured to establish at least the distal end of the secondary helix distal to a distal end of the housing when a force in the proximal direction is imparted on the secondary helix; and
processing circuitry electrically connected to the electrode, wherein the processing circuitry is configured to at least one of provide a signal or sense a signal using the electrode.

Example 33: The medical device of Example 32, wherein the electrode is one of a first electrode supported by the primary helix or a second electrode supported by the secondary helix.

Example 34: The medical device of Example 33, wherein the medical device includes the first electrode and the second electrode.

Example 35: The medical device of any of Examples 32-34, wherein the primary helix extends in the distal direction beyond the secondary helix.

Example 36: The medical device of any of Examples 32-35, wherein the primary helix defines a first number of turns around the longitudinal axis between the distal portion of the housing and a distal end of the primary helix and wherein the secondary helix defines a second number of turns around the longitudinal axis between the distal portion of the housing and a distal end of the secondary helix, wherein the second number of turns is less than or equal to 50% of the second number of turns.

Example 37: A method, comprising:
displacing, using a housing of a medical device, a first electrode within a tissue wall of a patient by imparting, to a primary helix supported by the housing, a torque in a first rotational direction about a longitudinal axis defined by the housing, wherein the primary helix extends in a distal direction beyond a distal portion of the housing, and wherein the primary helix supports the first electrode;
displacing, using the housing, a second electrode within the tissue wall by imparting, to a secondary helix supported by the housing, a torque in a second rotational direction about the longitudinal axis, wherein the secondary helix extends in the distal direction beyond the distal portion of the housing, and wherein the secondary helix supports the second electrode; and
at least one of providing or sensing, using processing circuitry, a signal using at least one of the first electrode or the second electrode.

Example 38: The method of Example 37, further comprising supporting, using a distal portion of the primary helix, the first electrode.

Example 39: The method of Example 37 or Example 38, further comprising supporting, using a distal portion of the secondary helix, the second electrode.

Example 40: The method of any of Examples 37-39, further comprising:
defining, using the primary helix, a first distance from a distal end of the primary helix to a distal end of the housing; and
defining, using the secondary helix, a second distance less than the first distance from a distal end of the secondary helix to the distal end of the housing.

Example 41: The method of any of Examples 37-40, further comprising, using the torque in the first rotational direction, slidably translating the secondary helix over a surface of the tissue wall when the primary helix displaces the first electrode within the tissue wall.

Example 42: The method of any of Examples 37-41, further comprising compressing, using a force in a proximal direction opposite the distal direction on the secondary helix, the secondary helix toward the housing.

Example 43: The method of any of Examples 37-42, further comprising substantially maintaining, using a support member supported by the housing, the distal end of the secondary helix beyond the distal end of the housing when a force in the proximal direction is imparted on the secondary helix.

Example 44: The method of Example 43, further comprising substantially maintaining, using a second portion of the support member, the distal end of the secondary helix beyond the distal end of the housing when the secondary helix contacts a first portion of the support member.

Example 45: The method of any of Example 43 or Example 44, further comprising displacing, using the tissue wall, the support member toward the housing when the tissue wall imparts a force in the proximal direction on the support member.

Example 46: The method of any of Examples 43-45, further comprising, using a deformation of the support member, a force in the distal direction on the secondary helix.

Example 47: The method of any of Examples 37-46, further comprising, using a proximal portion of the secondary helix, bending the secondary helix when the proximal portion of the secondary helix imparts a pulling force on a distal portion of the secondary helix.

Example 48: The method of any of Examples 37-47, further comprising, using the torque in the second rotational direction, pivoting the proximal portion of the secondary helix relative to the housing when the secondary helix exceeds a threshold travel within the tissue wall.

Example 49: The method of any of Examples 37-48, further comprising defining, using the primary helix, a first substantially circular helix with respect to the longitudinal axis, and defining, using the secondary helix, a second substantially circular helix with respect to the longitudinal axis.

Example 50: The method of any of Examples 37-49, further comprising, using a driver, at least one of the torque in the first rotational direction or the torque in the second rotational direction to a retrieval structure configured to transfer the torque to the housing.

Example 51: The method of any of Examples 37-50, further comprising displacing, using the housing, the first electrode within the tissue wall of a chamber of a heart of the patient.

Example 52: The method of any of Examples 37-51, further comprising, using the processing circuitry and at least one of the first electrode or the second electrode, at least one of providing the signal to the heart of the patient or sensing the signal from the heart of the patient.

Example 53: The method of any of Examples 37-52, further comprising transporting, using a delivery system, the housing through the vasculature of the patient.

What is claimed is:

1. A medical device configured to be positioned within an anatomical volume defined by a body of a patient, the medical device comprising:
a primary helix surrounding a longitudinal axis defined by a housing of the medical device, wherein the primary helix extends in a distal direction beyond a distal portion of the housing, wherein the primary helix supports a first electrode, and wherein the primary helix is configured to displace the first electrode within a tissue wall of a patient when a torque in a first rotational direction about the longitudinal axis is imparted to a housing;
a secondary helix surrounding the longitudinal axis, wherein the secondary helix extends in the distal direction beyond the distal portion of the housing, wherein the secondary helix supports a second electrode, and wherein the secondary helix is configured to displace the second electrode within the tissue wall when a torque in a second rotational direction about the longitudinal axis is imparted to the housing; and
processing circuitry electrically connected to the first electrode and the second electrode, wherein the processing circuitry is configured to at least one of provide a signal or sense a signal using at least one of the first electrode or the second electrode.

2. The medical device of claim 1, wherein a distal portion of the primary helix supports the first electrode.

3. The medical device of claim 1, wherein a distal portion of the secondary helix supports the second electrode.

4. The medical device of claim 1, wherein:
the distal portion of the housing defines a distal end of the housing,
the primary helix extends defines a first distance from the distal end of the housing to the distal end of the primary helix,
the secondary helix extends defines a second distance from the distal end of the housing to the distal end of the secondary helix,
the first distance and the second distance are substantially parallel to the longitudinal axis, and
the first distance is greater than the second distance.

5. The medical device of claim 1, wherein the secondary helix is configured to slidably translate over a surface of the tissue wall when the torque in the first rotational direction is imparted to the housing and the primary helix displaces the first electrode within the tissue wall.

6. The medical device of claim 1, wherein the primary helix defines one of a right-handed helix or a left-handed helix and the secondary helix defines the other of the right-handed helix or the left-handed helix.

7. The medical device of claim 1, wherein the secondary helix is configured to compress toward the housing when a force in a proximal direction opposite the distal direction is imparted on the secondary helix.

8. The medical device of claim 1, wherein the primary helix defines a first number of turns around the longitudinal axis between the distal portion of the housing and the distal end of the primary helix and wherein the secondary helix defines a second number of turns around the longitudinal axis between the distal portion of the housing and the distal end of the secondary helix, wherein the second number of turns is less than or equal to 50% of the second number of turns.

9. The medical device of claim 1, further comprising a support member supported by the housing, wherein the support member is configured to cause at least the distal end of the secondary helix to displace in the distal direction beyond the distal end of the housing when a force in the proximal direction is imparted on the secondary helix.

10. The medical device of claim 1, wherein the secondary helix includes a proximal portion supported by the housing, and wherein the secondary helix is configured to bend when the torque in the second rotational direction is imparted to the housing and the proximal portion imparts a pulling force on the distal portion.

11. The medical device of claim 10, wherein the proximal portion is configured to pivot relative to the housing when the torque in the second rotational direction is imparted to the housing and the secondary helix exceeds a threshold travel within the tissue wall.

12. The medical device of claim 1, wherein the primary helix defines a first substantially circular helix with respect to the longitudinal axis and the secondary helix defines a second substantially circular helix with respect to the longitudinal axis.

13. The medical device of claim 1, wherein the primary helix defines first pitch with respect to the longitudinal axis and the secondary helix defines a second pitch with respect to the longitudinal axis, and wherein the second pitch is greater than or equal to 75% of the first pitch and less than or equal to 125% of the first pitch.

14. The medical device of claim 1, wherein the primary helix is between the longitudinal axis and the secondary helix.

15. The medical device of claim 1, wherein the secondary helix is between the longitudinal axis and the primary helix.

16. A medical device configured to be positioned within an anatomical volume defined by a body of a patient, the medical device comprising:
a primary helix surrounding a longitudinal axis defined by a housing of the medical device, wherein the primary helix extends in a distal direction beyond a distal portion of the housing, and wherein the primary helix is configured to displace a distal end of primary helix within a tissue wall of a patient when a torque in a first rotational direction about the longitudinal axis is imparted to a housing;
a secondary helix surrounding the longitudinal axis, wherein the secondary helix extends in the distal direction beyond the distal portion of the housing, and wherein the secondary helix is configured to displace a distal end of the secondary helix within the tissue wall when a torque in a second rotational direction about the longitudinal axis and opposite the first rotational direction is imparted to the housing,
wherein at least one of the primary helix or the secondary helix supports an electrode;
a support member supported by the housing, wherein the support member is configured to establish at least the distal end of the secondary helix distal to a distal end of the housing when a force in the proximal direction is imparted on the secondary helix; and
processing circuitry electrically connected to the electrode, wherein the processing circuitry is configured to at least one of provide a signal or sense a signal using the electrode.

17. The medical device of claim 16, wherein the electrode is one of a first electrode supported by the primary helix or a second electrode supported by the secondary helix.

18. The medical device of claim 16, wherein the primary helix extends in the distal direction beyond the secondary helix.

19. A method, comprising:
displacing, using a housing of a medical device, a first electrode within a tissue wall of a patient by imparting, to a primary helix supported by the housing, a torque in a first rotational direction about a longitudinal axis defined by the housing, wherein the primary helix extends in a distal direction beyond a distal portion of the housing, and wherein the primary helix supports the first electrode;
displacing, using the housing, a second electrode within the tissue wall by imparting, to a secondary helix supported by the housing, a torque in a second rotational direction about the longitudinal axis, wherein the secondary helix extends in the distal direction beyond the distal portion of the housing, and wherein the secondary helix supports the second electrode; and
at least one of providing or sensing, using processing circuitry, a signal using at least one of the first electrode or the second electrode.

20. The method of claim 19, further comprising using the torque in the first rotational direction, slidably translating the secondary helix over a surface of the tissue wall when the primary helix displaces the first electrode within the tissue wall.
